(19) United States
(12) Patent Application Publication
(10) Pub. No.: US 2024/0120114 A1
(43) Pub. Date: Apr. 11, 2024

(54) MEDICINE EVALUATION SYSTEM

(71) Applicant: TALKING MEDICINES LIMITED, Glasgow (GB)

(72) Inventor: Elizabeth Anne Lyle BALMFORTH, Edinburgh (GB)

(21) Appl. No.: 18/264,678

(22) PCT Filed: Feb. 8, 2022
(86) PCT No.: PCT/GB2022/050333

§ 371 Date: Aug. 8, 2023

(30) Foreign Application Priority Data
Feb. 9, 2021 (GB) 2101783.5

(51) Int. Cl. G16H 70/40; G06F 40/169; G06F 40/295; G16H 10/20; G16H 15/00; G16H 50/70

(52) U.S. Cl. CPC G16H 70/40; G06F 40/169; G06F 40/295; G16H 10/20; G16H 15/00; G16H 50/70

(57) ABSTRACT

A method of estimating the effectiveness or safety of a medicine, the method including receiving commentary data encoding a plurality of items of commentary substantially related to medical subject-matter; processing the commentary data using at least one classifier to identify for each item a commentary type and a list of medicines associated with the commentary; selecting a subset of items, from the plurality of items of commentary, identified as referencing the medicine and whose commentary type has been identified as commentary from a patient who has used the medicine; processing the subset of items to generate content analysis data including, for each item, at least one estimate quantifying a respective at least one aspect of an effect of the medicine as described by the patient in the commentary; and processing the content analysis data to calculate an estimate indicative of the overall effectiveness or safety of the medicine.

The present invention relates to a method and system for evaluating medicine, and in particular relates to a method and system of evaluating the effectiveness or safety of a specific medicine or class of medicines.

### BACKGROUND TO THE INVENTION

Medicine manufacturers and medical professionals need to know that medicines that they manufacture and prescribe are effective and safe. Medical trials are a key part of this process, but they do not provide a complete answer. Once a medicine is in widespread use after being approved, unforeseen problems may arise with either effectiveness or safety. The confidence of patients in medicines may be shaken by real or perceived issues with specific medicines, leading to a reduced uptake of necessary medication with a negative clinical outcome. Thus, patient confidence in medicines and the effectiveness and safety of medicines are all important and interlinked.

There is also a desire more effectively to personalise a medicine treatment regimen for a particular patient. The better that a treatment can be personalised, the safer and more effective it will be for that patient. It is difficult to do so, however, without a good knowledge of how existing medicines and treatment regimens are currently being experienced by patients, and how effective and safe they appear to be. Such personalisation typically may include interventions to enable an individual to feel better and more in control as their disease state progresses from diagnosis to disease management.

There are different routes for medical professionals to pass on reports of patient experiences and problems that arise with medicines, but it has been found that this is not a sufficient mechanism to identify issues with specific medicines, nor to gain a statistically useful insight into the effectiveness or safety of medicines when in widespread use. One problem is that patients may not reliably report concerns or experiences to medical professionals, for example due to unwillingness or lack of contact; there is also a tendency for patients to respond to enquiries with answers that they think they should say. Another problem is that medical professionals may not pass on all relevant information for whatever reason.

In more recent times, it has become technically possible to provide apps or websites that allow either medical professionals or patients to report issues with medicines directly, but it has been found that this process is not taken up sufficiently, and that, for whatever reason, not all relevant information is reported accurately or at all.

In some cases it is possible for pharmaceutical companies to make contact with patients yet more directly, via face-to-face interviews and the like, to get direct feedback on the effectiveness or safety of the medicines that they are taking, but the cost of doing so may be prohibitive for a suitably large sample, and care must be taken not to violate laws regarding direct promotion of pharmaceutical products (and, furthermore, pharmaceutical companies regard health professionals as the consumers in the context of medicines, so they are not directly listening to patients). In this context, there remains the problem that patients tend to give answers that they believe are expected from them in response to a direct enquiry. The focus has therefore been on getting better feedback from medical professionals, but this is unsatisfactory for the reasons given above, and it remains a significant challenge to personalise medicines for particular patients effectively and safely.

The present invention seeks to address problems such as these in the prior art.

### SUMMARY OF THE INVENTION

In a first aspect of the invention there is provided a method of estimating the effectiveness or safety of a medicine, the method comprising: optionally receiving commentary data encoding a plurality of items of commentary substantially related to medical subject-matter; preferably, but not necessarily, processing the commentary data using at least one classifier to identify for each item a (apparent) commentary type and a list of medicines (apparently) associated with the commentary; as a further optional step, selecting a subset of items, from the plurality of items of commentary, identified as referencing the medicine and whose commentary type has been identified as commentary from a patient who has used the medicine; yet further, optionally, processing the subset of items to generate content analysis data including, for each item, at least one estimate quantifying a respective at least one aspect of an effect of the medicine described in the commentary (preferably about the medicine); and preferably processing the content analysis data to calculate an estimate indicative of the overall effectiveness or safety of the medicine. The latter estimate is preferably a summary score, and may be an estimate of patient confidence, which may be known as a Patient Confidence Score (PCS).

Preferably the commentary includes free text and/or unstructured data or text, such as text in any appropriate machine-readable encoding scheme. The commentary may specifically include or consist of social media posts (preferably extracts from such posts that exclude clearly superfluous or irrelevant portions such as headers and signatures), such as (specifically) Reddit® posts, Twitter® tweets, generic forum posts, audio and/or video podcasts, TikTok videos, YouTube videos, and so on. The commentary may additionally or alternatively be provided in e-mails, instant messages, and so on, and may as appropriate be manually or automatically transcribed or converted from other formats, such as audio, video, sign language, and so on.

The quantitative estimate of at least one aspect of an effect of the medicine may be considered a measure of the tone, preferably connoting a measure indicative of a linguistic and/or conceptual tone of the commentary, and/or a detected stance, preferably connoting a measure of a position that appears to be taken by the patient on any appropriate axis of sentiment or conceptual degree, for example as a measure of: how calm/angry; a level of anxiety; how neurotic/grounded; how happy/sad; or how confident/uneasy, and so on, that the patient appears to be as an effect of the medicine, whether directly or indirectly). Thus the quantitative estimate may include a sentiment estimate as mentioned below. The aspect of an effect of the medicine may in some cases be simply an effect of the medicine. The effect may be direct or indirect, as appropriate, but in some cases only direct (and in others only indirect).

The estimate may be quantifiable in any appropriate fashion, for example in terms of being positive or negative or just being an absolute value, and either quantifiable on a continuous (for example numeric) scale, or as a set of discrete values (such as positive, neutral, mixed, negative, highly positive, slightly positive, and so on). For example, a commentary may be analysed to estimate an aspect of the patient experience relating to a degree of approval (or disapproval) expressed about a particular entity, or as conveyed by the commentary in general terms without specific reference to any entity. The effect in question may be a primary effect, a side effect, or any other appropriate effect. Some examples are given below.

Preferably the term ‘classifier’ connotes a computerised model which is trained at least in part with training data of an appropriate type, and outputs a specific list of identifiers in response to receiving a specific input. Preferably a ‘classifier’ employs machine learning of any appropriate type to identify patterns in the input data and preferably to associate the patterns with specific corresponding outputs. A classifier may or may not be trained with data including annotations, tags or other manually identified features identifying relationships between specific examples of input data and associated features, concepts or entities, and the like. Any reference herein to a classifier identifying a specific thing should be considered, where appropriate, to relate to a classifier merely estimating a relationship to that thing, and/or identifying merely the appearance of that thing. One skilled in the art will understand that a classifier will not in general identify all aspects correctly, and that there is the possibility of a false positive or false negative (measured, essentially, by the concepts of precision and recall). Thus any mention of identifying features should not be considered to imply identifying features with 100% precision, for example.

The content analysis data may for example comprise textual analysis data, or may in some aspects further or alternatively comprise visual analysis data and/or audio analysis data, and so on (in the foreseen case where the items of commentary are not partly or wholly text-based, and may instead or additionally comprise audio and/or visual content).

It was discovered that, despite the high levels of knowledge that medical professionals and scientists may possess regarding the formulation and operation of various medicines, some of the most useful information regarding their efficacy and safety can be obtained from the patients who use those medicines, despite difficulties that have previously been experienced in obtaining that information. The use of at least one classifier to identify a patient commentary type facilitates the selection of this more informative data without requiring any direct contact with patients or any manual/human intervention during the search process itself.

It was furthermore discovered that it was in general easier and more economical to obtain useful information on medicines from those patients via unstructured text, rather than by directly questioning the patients in a structured way. It has been found that patients are more likely to communicate more honestly and openly about their experiences with their own peer group. It was found that patients often use social media to explore symptoms and medicines and to share their experiences (often anonymously, increasing candidness, which is not possible in direct consultations).

It was also discovered that using at least one classifier to identify a list of medicines was advantageous relative to other methods such as (only) simple keyword searching, for example because of the false matches that may arise due to some ordinary words being used for names of medicines, and some matches being missed because of simple misspellings and the like.

Even yet furthermore, it was found that the use of at least one classifier to identify both the commentary type and the medicines mentioned in the commentary provided an unexpected synergy, because it was found that the way that medicines were referred to depended on the commentary type, and in particular the author type; for example medical professionals typically refer to medicines differently to patients who are prescribed those medicines.

Overall, these advantageous features can allow the effectiveness and safety of medicines to be scrutinised more effectively, amongst other things allowing an improved personalisation of medicines for individual patients.

Preferably the method may be extended to a plurality of medicines and/or one or more classes of medicine. The method may further comprise identifying at least one symptom or side effect and identifying the medicine (or plurality of medicines or one or more classes of medicine) in dependence on its association with said at least one symptom or side effect.

In general, connections are drawn between at least two of the three groups of entities comprising: (1) patient persona, feelings and lifestyle; (2) symptoms and severity of disease or symptoms; and (3) a health condition or infection. It will be appreciated that references herein to one entity in a group may be replaced as appropriate with any other entity within the same group.

The plurality of items may be pre-filtered by any appropriate method, for example to select items in a particular language (such as English) or set of languages, and/or to select items by geographical location (for example by geo-mapping IP addresses on social media posts or using user-supplied geographical information).

Preferably the method further comprises downloading the plurality of items of commentary from remote data sources such as websites on the Internet or databases connected to apps or other computer programs, preferably using Application Programming Interfaces (APIs) provided by the operators of the websites, apps, and so on, and preferably the method further comprises processing the plurality of items of commentary to normalise the format of the items, for example including removing headers, footers, signatures and/or other irrelevant or unnecessary data; removing invalid characters; converting into a unified text encoding scheme, and so on. Preferably the method further comprises storing the normalized items of commentary in an intermediate data store, for example in a data store within the computer system carrying out at least part of the processing.

Preferably said at least one classifier includes a Named Entity Recognition, NER, classifier for identifying at least the list of medicines associated with (that is, preferably referenced by) the commentary. A Named Entity Recognition classifier can provide considerable flexibility and can take context into account and account for misspellings and so on. The term ‘NER classifier’ is preferably used in the sense of ‘classifier’ described above, for example taking a data source to be analysed as an input, and producing any number of identified features of the input data source as an output. The term is preferably intended to cover ‘NER recognisers’, ‘NER models’ (as such), and so on, providing the appropriate functionality is provided.

The NER classifier additionally identifies references to at least one of: type of medicine, ailment, condition, symptom, potential side effect, possible medical outcome, and treatment type. Preferably at least one said classifier further identifies personal experiences or feelings described by the patient. This task may also be performed by the NER classifier as aforesaid, for example, or could be performed by a separate classifier that may specialise in this task.

Processing the subset of items to generate content analysis data preferably further comprises processing the personal experiences or feelings described by the patient.

Said at least one quantitative estimate of at least one aspect of an effect of the medicine preferably includes an estimate of at least one of: a perceived effectiveness of the medicine, a perceived safety of the medicine, happiness or unhappiness associated with the medicine, and satisfaction or dissatisfaction associated with the medicine. It was found that in general a correlation could be drawn between these aspects of the patient experience and the overall effectiveness or safety of a medicine. Therefore patient commentary can be turned into concrete, useful technical information that can be used to help personalise medicine for patients, by (for example) accessing the right medicine, in the right treatment pathway, to get the right dosing amounts and/or dosing schedules, at the right time. In some cases the insights provided by the present method can allow medicine producers (or anyone else) to re-evaluate and/or improve the formulation of medicines. The present method can potentially also assist clinical trials, for example, by providing early/additional insights that may assist decision making in the trials.

Processing the subset of items to generate content analysis data preferably further comprises processing (for example, with a sentiment tagging processor) the subset of items to calculate a sentiment estimate, encoding a measure of at least one of: positivity, negativity and neutrality, expressed by the patient in relation to at least one of: the commentary considered as a whole, each mention of the medicine individually, and every mention of the medicine considered as a whole. Any aspect of this feature can be provided independently of the other.

In the event that multiple estimates of positivity or negativity are calculated, an overall estimate may be selected based on at least one of: an average of the individual estimates, a predominant one of the individual estimates (that is, for example, the numerically largest estimate), or an individual estimate selected on any appropriate characteristic. A separate or alternative estimate may be calculated of how mixed or balanced each aspect of an effect of the medicine is.

The method may further comprise dividing each item (such as a social media post) into at least one part (such as an individual sentence or other partial fragment of the post), wherein processing the subset of items to calculate a sentiment estimate may comprise processing each part (such as a sentence) separately, and wherein each sentiment estimate relates to a respective part (rather than, optionally, the post as a whole, for example). The sentiment estimate may alternatively relate to either the post or a part of the post as appropriate. The method may further comprise processing each such part to determine whether or not to exclude the part from further processing. Thus, the method may involve only considering a subset of the identified parts in further processing, allowing less relevant or misleading material to be excluded from the further processing.

The method may further comprise processing each of the subset of items to identify at least one specific characteristic in the item and, if a said specific characteristic is identified, to exclude the item from further processing. This is preferably in addition to and distinct from any of the classification steps mentioned above. The characteristics may include at least one of: post category (such as subreddit or similar), author (or author type), keywords, and key phrases. The characteristics may be associated with irrelevant types of posts, for example posts created by bots of various kinds. The combination of classification techniques with more deterministic filtering techniques can provide an optimal process for focusing on more relevant material.

Preferably said at least one classifier includes a commentary type classifier. Alternatively, the Named Entity Recognition classifier as aforesaid, or any other appropriate classifier, can identify the commentary type (for example, only a single classifier may be used). This form of ‘multitask’ classifier can provide improved accuracy by being able more closely to take into account correlations between commentary (and especially author) type and other features identified by the classifier, for example. Multiple classifiers can be more efficiently implemented and trained, however.

The commentary type classifier is preferably configured to identify at least one commentary type, selected from at least one of: patient opinion, medical reference data, medical professional opinion, scientific report (such as an official scientific journal entry, individual scientific publication, or science blog, and so on), industry report (for example professional or other reports from the medicine manufacture or medicine research sectors), news report (such as a written report, a video or audio report, or transcription of a video or audio report), and structured feedback (for example via an experience-reporting app or website for use by patients, doctors, others, or any appropriate combination thereof).

The commentary type classifier is more preferably configured to identify at least one author type, selected from at least one of: patient (preferably someone who has used the medicine), medical professional, medicine representative, research scientist, journalist, and other type. The ‘other’ type may be one or more types not mentioned above, and may in particular be a type defined (simply) as being not one of the defined types, and the like.

Preferably the commentary type classifier is trained or trainable to detect at least the following author types: patient, carer for a patient, medical professional (specifically at least one of a doctor, nurse, general practitioner, senior doctor, research doctor, student, or other, for example), someone involved in the production of a medicine, someone involved in research into new medicines, someone involved in the marketing or management of a medicine, and so on. Any appropriate subset can be discriminated between, so long as an appropriate patient author type (or multiple patient author types) is trained for and/or identified. The author type classifier may be known as a patient voice detector or similar.

Preferably a plurality of author types and/or a plurality of commentary types are identified. By configuring the at least one classifier to recognize a plurality of commentary and/or author types (including commentary types and author types that are not desired to be selected).

In this case, selecting the subset of items may further comprise filtering out (that is, excluding from further processing, or otherwise limiting the use of in some way) author types being identified as other than patient. In one aspect, the commentary type classifier may output multiple identifications, for example with associated confidence estimates or ranges, or otherwise, and selecting the subset of items may further comprise at least one of: filtering out items having at least one author type other than patient, selecting items with at least one author type of patient, and selecting items where a measure of confidence that the author type is patient is greater than a measure of confidence that the author type is a type other than patient. That is, selecting the subset of items may comprise excluding commentary by medical professionals; excluding commentary by news outlets, and so on.

Preferably at least one said at least one classifier is selected in dependence on the medicine. Thus, the classifier may be selected directly in dependence on the medicine, or in dependence on at least one of (for example): a class of medicines to which the medicine belongs, a treatment area covered by the medicine, or side effects associated with the medicine, and so on. Accordingly, there may be provided classifier data encoding a plurality of classifiers (or underlying models) of the same type (whether NER or otherwise), each of the plurality of classifiers is associated with a different medicine, class of medicine, or area of medicine and/or treatment. Said at least one classifier may thus be selected for use, in relation to a single item of commentary or otherwise, based on a specified medicine, class of medicine, area of medicine and/or treatment, or in accordance with any other relevant or appropriate entity.

In one aspect, multiple sets of classifiers may be provided if, for example, multiple medicines or a group of medicines are selected for analysis, or if a single medicine covers multiple areas of treatment, or multiple symptoms, and so on.

In a more sophisticated solution, at least one NER classifier is provided per at least one of: area of medicine, type of medicine, specific medicine, ailments, conditions, treatments, potential outcomes, and potential side effects. This can provide improved results considering that the style of commentary was found to vary depending on area of medicine (and consequently on related features such as conditions, symptoms, treatments, and so on). Accordingly, a plurality of data sets may be stored in a database, encoding a respective plurality of NER classifiers.

In one aspect, selecting the subset of items further comprises filtering the subset of items by one of a plurality of cohorts, and calculating a respective estimate indicative of the overall effectiveness or safety of the medicine for each cohort. A cohort preferably connotes a group of individuals having a statistical factor (such as age or class membership) in common, but preferably relates to any appropriate division of the entire class of patients into two or more groups by any appropriate factor, including but not limited to age, biological sex, (apparent) income, previous medical history, geographic location, disease progression, symptoms, side effects, treatments received, and so on. The method may further comprise using the output of said at least one classifier and/or the output of a further at least one classifier in order to identify to which cohort of patient each item of commentary appears to relate.

Thus, by producing a plurality of indicative estimates for respective cohorts of the patient population, a number of advantages arise: firstly, it is possible to identify cohorts where, for whatever reason, use of a particular medicine is problematic, which can allow targeted intervention to improve effectiveness and safety, for example by adjusting dosage or choice of medicine for that particular cohort, or initiating a clinical trial with a more limited (and therefore more economical) focus. By analysing the patient commentary by cohort, it is also easier to provide personalised medicine, and to create useful correlations between the experiences of patients in one cohort (say, young people with early onset of disease) between the experiences of patients in another cohort (say, older patients), to facilitate providing an optimal medicine regimen to maximise quality of life for the longest possible time.

It is also possible to subdivide the subset of items in any appropriate fashion, for example by data source. Since some data sources (principally social media sources) appear to be more clinically-focused, and other data sources (principally medicine management apps) appear to be more community-focused, a comparison of the resulting indicative estimates can provide useful insights into medicine use and disease progression.

In one aspect, a summary score can be provided for any selected division or filtering of a particular medicine classification structure, for example for each of a set of medicines, at least one class of medicines, at least one mode of action, at least one therapeutic indication, at least one pharmaceutical company, at least one country, and so on.

The method preferably further comprises: accessing a medicine database containing medicine data that encodes a plurality of medicine names associated with at least one jurisdiction; retrieving data from the medicine database in accordance with a search query, the retrieved data including at least one medicine name, wherein selecting the subset of items further comprises processing (only) items of commentary which include at least one said at least one medicine name.

Preferably the search query specifies at least one jurisdiction to filter by, whereby only medicine names relevant to the, or each, specified jurisdiction will be provided. In one aspect, the medicine data additionally includes at least one of: symptoms treated by a medicine, potential side effects of a medicine, areas of medicine relevant to a medicine, types of treatment related to a medicine, marketer of a medicine, manufacturer of a medicine, licensee and/or licensor of a medicine, and so on. In this case, the retrieved data may include at least a portion of this additional data. Alternatively or additionally, the search query may be directed to at least one of these additional data types, for example allowing medicine names to be output for all medicines being used to treat a particular condition, or outputting medicine names for all medicines produced by a particular drug company, and so on. Preferably searches are limited to a small number of medicines, and ideally one medicine, however, to produce more granular outputs.

This optional feature can be used to pre-filter the data inputted to the at least one classifier. The at least one medicine name preferably includes at least one of: a local trading name for a medicine in a particular said jurisdiction (such as Nurofen®), a generic name for a medicine (such as ibuprofen), a brand name for a medicine (such as Advil), a brand name of a supplier of a medicine (for example if a supplier is predominantly known for the medicine), and optionally a name of a class of medicines including a medicine (such as analgesic and/or non-steroidal anti-inflammatory drug, NSAID), in some cases only if the class of medicines is used by patients in a way that is substantially synonymous with a specific medicine, for example.

It will be appreciated that a post which was downloaded because it contained a medicine keyword from the medicine filter list may ultimately be rejected because the at least one classifier did not identify a valid medicine identifier. This may happen, for example, if the name in question was not used in a medical context (for example, if a post was downloaded because it contained the name ‘Nikki’, but the word ‘Nikki’ was used in the post as a person's name, not as a medicine name). It will also be appreciated that pre-filtering the data may create false negatives for references to medicines which are valid but do not match the data in the medicine database, for example due to misspellings or due to out-of-jurisdiction medicine names being used.

A decision on whether or not to pre-filter based on medicine names can be taken based on the specific circumstances of the various data sets, and so on. Alternatively or additionally, the data inputted to the at least one classifier can be pre-filtered (or initially selected) by manually or automatically selecting (only) from groups of commentary such as sub-forums, sub-reddits, community groups, hashtags, and so on, and optionally ignoring commentary in other sub-forums, sub-reddits, and so on.

Typically, said plurality of items are initially selected by performing a plurality of searching or filtering operations to find a respective plurality of sets of items of commentary, and forming the plurality of items from the plurality of sets of items of commentary. The searching or filtering may be a keyword search or may be a more complicated search query in any appropriate form.

Preferably the sets of items of commentary are combined as a union set, whereby all unique results are taken from all of the searches/filter operations. Alternatively, the intersection of the different sets can be taken, where only results appearing in all search results are taken. The method used may instead be an appropriate combination of these two methods, or any other merge operation as appropriate. The appropriate combination taken is preferably determined with reference to the specific area of medicine or treatment, and so on. One area of medicine may require a relatively large number of specific searches/filters to be used, and in another area of medicine it may be possible to capture most results of interest using relatively few searches/filter operations.

The aforementioned searching or filtering operations preferably include searching or filtering by medicine name, optionally including alternative names for the same medicine or active ingredient. The searching or filtering operations may further optionally or additionally comprise filtering or searching by at least one of: commentary type, author type, type of medicine, ailment, condition, symptom, potential side effect, possible medical outcome, treatment type, personal experience, measure of the tone, detected stance, and feeling or emotion. Preferably substantially all of the symptoms, side effects, treatment types and so on are related to the medicine.

Processing the content analysis data preferably further comprises: processing a plurality of feature indicators selected from at least one of: at least one aspect of an effect of the medicine, at least one aspect of the patient experience, at least one measure of tone of the commentary (if otherwise), at least one detected stance of the patient, a count of the number of times a medicine is mentioned, a count of the number of times a relevant symptom is mentioned, a sentiment estimate, and a count of the number of times a relevant feeling or experience is mentioned; and combining the indicators to generate the estimate indicative of the overall effectiveness or safety of the medicine.

Preferably the indicators are numeric or otherwise quantified, and more preferably the indicators are normalized in an appropriate fashion to facilitate their combination, for example by taking a mean average, a median average, other form of average, or any appropriate combination of these, or may be calculated or derived using a more complicated algorithm or model, as appropriate.

Preferably calculating the estimate indicative of the overall effectiveness or safety of the medicine comprises selecting a set of the feature indicators, applying a respective weighting to each selected feature indicator, and combining the weighted plurality of feature indicators into a single estimate indicative of the overall effectiveness or safety of the medicine. Appropriate selection of features and weighting thereof can avoid any one factor having a disproportionate effect on the overall estimate.

Preferably the selected set of feature indicators includes at least one primary indicator of effectiveness or safety of the medicine (such as the sentiment estimate) and at least one relevance indicator, representing a measure of the amount of opinion expressed (including, for example, any or all of: medicine count, feeling count, symptom count and brand count). The use of relevance indicators can help to avoid giving undue weight to relatively statistically insignificant data relating to the primary indicator(s).

The method may yet furthermore comprise restricting at least one of the selected feature indicators to a sub-range within the output range of the single estimate. For example, the primary indicator and/or the sentiment estimate in particular may be limited so that the primary indicator cannot by itself cause the ultimate estimate to reach extreme positive or negative values. For an output estimate range between 0 and 1 (say), the sub-range may for example be between 0.1 and 0.9, or between 0.2 and 0.8, and so on.

Processing the content analysis data is preferably carried out with respect to a predetermined time period, wherein the subset of items is selected in respect of commentary falling within the predetermined time period.

Preferably the commentary is deemed to have been written within the appropriate time period, but may alternatively (as appropriate) have been published and/or collected within the time period. The time period may be any appropriate measure of time, such as a day, week, month, year, and so on, and may be a regular time period, or simply a time period defined as the time between one sampling of data and the next. The collection of the commentary data may be carried out on an essentially continuous or irregular basis, or may be triggered on a regular basis corresponding to the length of the time period. In the event that no relevant commentary data exists, or a statistically insufficient amount of data exists, a default effectiveness or safety rating may be provided, and/or the rating may be marked as a null (that is, an invalid rating which may be excluded from further analysis or graphing). In one embodiment, the predetermined time period is an integer multiple of years, in order to reduce the effect of seasonal variations.

Preferably the method further comprises: selecting one of the plurality of items of commentary; providing annotation data associated with the selected item of commentary, the annotation data identifying at least one (tag) of: commentary type, author type, name of medicine, type of medicine, ailment, condition, symptom, potential side effect, possible medical outcome, treatment type, aspect of a personal experience, measure of the tone (of the commentary), detected stance (of the patient), and feeling or emotion; and training or retraining said at least one classifier using the combination of the selected item of commentary and the associated annotation data. Preferably the annotation data identifies at least one location in the selected item of commentary relating to at least one tag (as enumerated above), and preferably identifies a location in the item of commentary for every identified tag (or other feature). Put another way, preferably the annotation data identifies at least one relationship between a portion of the selected item of commentary and an associated entity or feature. That is, the annotation data may include at least one tag, corresponding to an entity or feature, and at least one location within the selected item of commentary. Changes to the annotation data may for example include adding, deleting or modifying tags and/or adding, deleting or modifying associated locations in the selected item of commentary.

This provides a mechanism for improving the selection of items of commentary from the classifier(s). Preferably the annotation data comprises at least a list of tags, preferably identifying matching pairs of entity class and entity instance, such as “Feeling: Angry”, “Medicine: Lipitor®”, or “Symptom: Heartburn”, for example) and preferably the annotation data includes location data identifying which specific parts of the commentary relate to which specific tags. In that case, preferably outputting the annotation data comprises displaying the item of commentary with the annotation tags overlaid in any appropriate form, such as highlighting parts of text and/or drawing connections between parts of text and displayed tags.

Preferably the method yet further comprises: outputting the selected item of commentary to an annotation user; optionally, if it is present, outputting the annotation data to the annotation user; receiving user input from the annotation user including a direction to create, modify or delete at least a portion of the annotation data; and creating, modifying or deleting at least a portion of the annotation data in accordance with the received direction. The term ‘annotation user’ preferably connotes any user who may be involved in a relevant interaction and may include, but is not limited to, annotators. A client user, who may be the same person or class of person or otherwise, may be involved in receiving or accessing the outputted estimate indicative of the overall effectiveness or safety of the medicine.

Thus a manual annotation process can be introduced, allowing the creation of annotation data for a particular item of commentary, as a basic step in training the classifier(s), and/or the refinement of existing annotation data to provide improved training for the classifier(s). Alternatively (or selectively) automated machine learning methods may be used to train the classifier(s) using live sets of data or separate training sets of data. Outputting existing annotation data can allow quality control of the training of the at least one classifier, which can increase the accuracy of the, or each, classifier, and optionally provide a ‘gold standard’ dataset for model optimisation. It will be appreciated that for previously un-annotated items of commentary, the annotation data will be empty/null, in which case the step of outputting the annotation data need not involve any actual process steps.

In a related aspect, the method comprises processing the commentary data for the selected item using said at least one classifier; and providing the annotation data includes providing, at least in part, the output of said at least one classifier.

This can provide potentially useful initial suggestions when initially annotating an item of commentary. The process as aforesaid can also allow irrelevant items of commentary to be excluded before being annotated, reducing the workload of any annotators (human or otherwise). These steps can also allow enhanced quality control, allowing the output of the classifier(s) to be scrutinized and adjusted, and the classifier(s) retrained with the adjusted results, for example.

It will be appreciated that various of the above features provide a positive feedback loop where the commentary type classifier(s) is made more efficient because it is trained (only) on patient commentary type data, with other data filtered out, and the annotation process is improved because the annotation users see predominantly only patient commentary type data. This in turn improves the commentary type classifier(s), and so on. Put another way: the editing of annotation data and the identification of patient commentary types by the at least one classifier together enjoy a synergy that leads to an efficient annotation process and an improved classification of items of commentary, certainly compared to any hypothetical systems in which annotators have to work with items of commentary that have not previously been filtered by commentary type, or which have not been pre-populated with automatically identified ‘tags’, and the like.

The various features described immediately above may be also be provided in independent form. Thus in a related aspect, the method comprises processing a plurality of items of commentary with at least one classifier to generate annotation data for each item including a commentary type identifier and a number of entity identifiers; selecting a subset of the plurality of items of commentary for annotation that are identified as a patient commentary type; providing the selected subset of the plurality of items to an annotation user (as aforesaid); facilitating the editing of the annotation data (including creating, altering and/or deleting the data at least in part) by the annotation user; and (re)training said at least one classifier with the edited annotation data whereby to improve the identification of items having a patient commentary type, and consequently to facilitate the annotation of additional items of commentary. This method may also be provided without the first step of creating the annotation data (that is, to allow the editing of annotation data for initially unannotated items of commentary, which may include various commentary types).

The method preferably further comprises repeating at least one of the steps of: processing the commentary data using said at least one classifier, selecting a subset of items, processing the subset of items, and processing the content analysis data after training or retraining said at least one classifier with the new or modified annotation data.

In a related aspect of the invention, there is provided a method of training a selected classifier for use with a method as aforesaid (fully, partially, or in some cases not at all, as appropriate), wherein the selected classifier is configured to identify, for an item of commentary, at least one of a commentary type and a list of medicines associated with the commentary, and wherein the method comprises: processing the commentary data using at least one classifier, including said selected classifier, to identify for each item a commentary type and a list of medicines associated with the commentary; selecting one of the plurality of items of commentary; providing annotation data associated with the selected item of commentary, the annotation data identifying at least one of: commentary type, author type, name of medicine, type of medicine, ailment, condition, symptom, potential side effect, possible medical outcome, treatment type, aspect of a personal experience, measure of the tone, detected stance, and feeling or emotion; and training or retraining the selected classifier using the combination of the selected item of commentary and the associated annotation data.

As before (or otherwise) the method may further comprise outputting the selected item of commentary to an annotation user; outputting the annotation data to the annotation user; receiving user input from the annotation user including a direction to create, modify or delete at least a portion of the annotation data; and creating, modifying or deleting at least a portion of the annotation data in accordance with the received direction. The method may yet further comprise processing the commentary data for the selected item using the selected classifier; and wherein providing the annotation data includes providing, at least in part, the output of the selected classifier. Other method features may of course be provided as aforesaid and entirely as appropriate and/or possible.

In another aspect of the invention, there is provided a computer system for estimating the effectiveness or safety of a medicine, the computer system comprising: at least one processor and at least one associated memory store; wherein said at least one memory store includes computer program code which, when executed by said at least one processor, causes the computer system to perform the method of: optionally receiving commentary data encoding a plurality of items of commentary substantially related to medical subject-matter; preferably, but not necessarily, processing the commentary data using at least one classifier to identify for each item a (apparent) commentary type and a list of medicines (apparently) associated with the commentary; as a further optional step, selecting a subset of items, from the plurality of items of commentary, identified as referencing the medicine and whose commentary type has been identified as commentary from a patient who has used the medicine; yet further optionally processing the subset of items to generate content analysis data including, for each item, at least one estimate quantifying at least one aspect of an effect of the medicine described in the commentary (preferably about the medicine); and preferably processing the content analysis data to calculate an estimate indicative of the overall effectiveness or safety of the medicine.

The system may further comprise (or may simply have access to) a classifier database for storing classifier data encoding (the mathematical model of) at least one classifier, a commentary database for storing a plurality of items of commentary, and/or a medicines database for storing medicine data. No special technical limitation is implied for any of the databases beyond what is necessary or appropriate.

The classifier, commentary and/or medicine database may be embodied in any appropriate physical form. For example, a single physical data store may store more than one database as aforesaid, and/or a single database as aforesaid may be stored across a plurality of physical data stores (for example using cloud storage, RAID arrays, and the like). Multiple processors may be served by a single memory store, and single processors may be served by multiple associated memory stores, for example, which may partially or fully overlap with the database(s). The commentary data may not be stored at all (other than in the original format) and the identified data output by the at least one classifier may be stored instead of the original commentary. In another aspect, the commentary data is stored by the system and the identified data is generated on request and/or on-the-fly, which has the benefit that the most up to date trained version of the classifier(s) is used. In another aspect, both the original commentary data and the identified data are stored, and either can be updated as and when required. In this latter case, it is possible that not the most up to date identified data is used, but a calculation of the estimate of effectiveness or safety can be carried out much more quickly based on the pre-computed values that are stored.

In another aspect of the invention there is provided a computer system for training or retraining a classifier for use with a computer system as immediately aforesaid (or otherwise), wherein the selected classifier is configured to identify, for an item of commentary, at least one of a commentary type and a list of medicines associated with the commentary, and wherein the system comprises: at least one processor and at least one associated memory store; wherein said at least one memory store includes computer program code which, when executed by said at least one processor, causes the computer system to perform the method of: selecting one of a plurality of items of commentary; causing the commentary data for the selected item of commentary to be processed using the selected classifier; outputting the selected item of commentary to an annotation user; receiving annotation data associated with the selected item of commentary, the annotation data identifying at least one of: commentary type, author type, name of medicine, type of medicine, ailment, condition, symptom, potential side effect, possible medical outcome, treatment type, aspect of a personal experience, measure of the tone (of the commentary), detected stance (of the patient), and feeling or emotion, and the annotation data including, at least in part, the output of the selected classifier; outputting the annotation data to the annotation user; receiving user input from the annotation user including a direction to create, modify or delete at least a portion of the annotation data; creating, modifying or deleting at least a portion of the annotation data in accordance with the received direction; and causing the selected classifier to be trained or retrained using the combination of the selected item of commentary and the associated annotation data.

Preferably the steps of causing the commentary data to be processed and causing the selected classifier to be trained or retrained comprise transmitting a command either locally or remotely to cause the actions to be undertaken. One or more aspects of the aforesaid computer system may be implemented in distributed form, for example via cloud computing methods. Preferably the computer system is in networked communication with a central computer system which stores and/or accesses data stores including but not limited to a database of items of commentary, a database of data encoding the at least one classifier, and a database of medicine data, all as aforesaid or otherwise.

In a further aspect of the invention, there is provided a computer system for estimating the effectiveness or safety of medicines, the computer system including: a commentary downloader module for downloading items of commentary from at least one remote source; a commentary type classifier module for identifying the type of commentary; a named entity recognition, NER, classifier module for identifying entities associated with each item of commentary; a medicine database encoding medicine data that encodes a plurality of medicine names associated with at least one jurisdiction; a commentary importer module which accesses and applies the commentary type classifier module, the NER classifier module and the medicine data in the medicine database, to select from the downloaded items of commentary a plurality of items of commentary that include at least one medicine entity, that include at least one appropriate medicine name, and that are identified as being a commentary type that is authored by a patient; a feature calculator module configured to calculate for each item of commentary a plurality of feature indicators selected from at least one of: at least one aspect of an effect of the medicine, at least one aspect of the patient experience, at least one measure of the tone of the item of commentary, at least one detected stance of the patient, a count of the number of times a medicine is mentioned, a count of the number of times a relevant symptom is mentioned, a sentiment estimate, and a count of the number of times a relevant feeling or experience is mentioned; a summary score calculator module for calculating a summary score (corresponding for example to the indicative estimate as aforesaid) representative of the effectiveness or safety of a medicine in dependence on the feature indicators calculated by the feature calculator module for relevant items of the plurality of items of commentary. A method equivalent of this (and any other) system aspect is of course envisaged.

The computer system may optionally further include a central data store for storing the plurality of items of commentary and associated outputs of the classifier modules, and may yet further include a raw post store database, a model and/or gazetteer store, and a medicine database including medicine data as aforesaid.

The computer system may further comprise an annotation entry (computer) system configured to: receive an item of commentary; receive associated annotation data encoding the output of the commentary type classifier module and the NER classifier module in respect of the item of commentary; output the item of commentary and the associated annotation data; receive adjustments or additions to the annotation data; carry out the adjustments or additions to the annotation data; transmit the adjusted annotation data and cause at least one of the commentary type classifier module and the NER classifier module to be trained or retrained using the adjusted annotation data.

The annotation entry system may be part of the computer system as aforesaid or may comprise a separate (at least one) processor and associated memory and computer program code, for example in the form of a stand-alone or remote computer terminal.

In another aspect of the invention, there is provided a corresponding method of estimating the effectiveness or safety of medicines, comprising: downloading items of commentary from at least one remote source; applying a commentary type classifier and a named entity recognition, NER, classifier to select from the downloaded commentary a plurality of items of commentary that include at least one medicine entity and that are identified as being a commentary type that is authored by a patient; for each of the plurality of items of commentary, calculating a plurality of feature indicators selected from at least one of: at least one aspect of an effect of the medicine, at least one aspect of the patient experience, at least one measure of the tone of the item of commentary, at least one detected stance of the patient, a count of the number of times a medicine is mentioned, a count of the number of times a relevant symptom is mentioned, a sentiment estimate, and a count of the number of times a relevant feeling or experience is mentioned; and calculating a summary score representative of the effectiveness or safety of a medicine in dependence on the feature indicators calculated for relevant items of the plurality of items of commentary.

The method may further comprise: receiving an item of commentary; receiving associated annotation data encoding the output of the commentary type classifier and the NER classifier in respect of the item of commentary; outputting the item of commentary and the associated annotation data; receiving adjustments or additions to the annotation data; carrying out the adjustments or additions to the annotation data; transmitting the adjusted annotation data and cause at least one of the commentary type classifier and the NER classifier to be trained or retrained using the adjusted annotation data.

Any method as aforesaid may further comprise personalising a medicine regimen for a patient in dependence on at least one of: the estimate indicative of the overall effectiveness or safety of the medicine, said at least one estimate quantifying a respective at least one aspect of an effect of the medicine as described by the patient in the commentary, and at least one output of said at least one classifier.

In another aspect of the invention there is provided a method of estimating patient confidence in a medicine, the method comprising: (optionally) receiving commentary data encoding a plurality of items of commentary substantially related to medical subject-matter; preferably, but not necessarily, processing the commentary data using at least one classifier to identify for each item a (apparent) commentary type and a list of medicines (apparently) associated with the commentary; as a further optional step selecting a subset of items, from the plurality of items of commentary, identified as referencing the medicine and whose commentary type has been identified as commentary from a patient who has used the medicine; yet further optionally processing the subset of items to generate content analysis data including, for each item, at least one estimate quantifying a respective at least one aspect of an effect of the medicine described in the commentary (preferably about the medicine); and preferably processing the content analysis data to calculate an estimate of patient confidence in the medicine.

In a yet further aspect of the invention there is provided a method of estimating patient confidence in a medicine, comprising: downloading items of commentary from at least one remote source; applying a commentary type classifier and a named entity recognition, NER, classifier to select from the downloaded commentary a plurality of items of commentary that include at least one medicine entity and that are identified as being a commentary type that is authored by a patient; for each of the plurality of items of commentary, calculating a plurality of feature indicators selected from at least one of: at least one aspect of an effect of the medicine, at least one aspect of the patient experience, at least one measure of the tone of the item of commentary, a detected stance of the patient, a count of the number of times a medicine is mentioned, a count of the number of times a relevant symptom is mentioned, a sentiment estimate, and a count of the number of times a relevant feeling or experience is mentioned; and calculating a summary score representative of patient confidence in a medicine in dependence on the feature indicators calculated for relevant items of the plurality of items of commentary.

It will be appreciated that generally any feature as aforesaid which can allow the effectiveness and/or safety of a medicine to be assessed can also allow a patient confidence in the medicine to be assessed, and can be adapted accordingly.

Although the embodiments of the invention described above with reference to the drawings may comprise computer-related methods or apparatus, the invention may also extend to program instructions, particularly program instructions on or in a carrier, adapted for carrying out the processes of the invention or for causing a computer to perform as the computer apparatus of the invention. Programs may be in the form of source code, object code, a code intermediate source, such as in partially compiled form, or any other form suitable for use in the implementation of the processes according to the invention. The carrier may be any entity or device capable of carrying the program instructions.

Thus, there is specifically provided in a further aspect of the invention a non-transitory computer readable medium encoding computer program code which, when executed on at least one processor of a computer, causes the computer (or any appropriate combination of computers, with appropriate distribution of the computer program code) to carry out any appropriate method as aforesaid.

For example, the carrier may comprise a storage medium, such as a ROM, for example a CD ROM or a semiconductor ROM, or a magnetic recording medium, for example a floppy disc, hard disc, or flash memory, optical memory, and so on. Further, the carrier may be a transmissible carrier such as an electrical or optical signal which may be conveyed via electrical or optical cable or by radio or other means. When a program is embodied in a signal which may be conveyed directly by cable, the carrier may be constituted by such cable or other device or means.

Although various aspects and embodiments of the present invention have been described separately above, any of the aspects and features of the present invention can be used in conjunction with any other aspect, embodiment or feature where appropriate. For example apparatus features may where appropriate be interchanged with method features. References to single entities should, where appropriate, be considered generally applicable to multiple entities and vice versa. Unless otherwise stated herein, no feature described herein should be considered to be incompatible with any other, unless such a combination is clearly and inherently incompatible. Accordingly, it should generally be envisaged that each and every separate feature disclosed in the introduction, description and drawings is combinable in any appropriate way with any other unless (as noted above) explicitly or clearly incompatible.

### DESCRIPTION OF THE DRAWINGS

An example embodiment of the present invention will now be illustrated with reference to the following Figures in which:

FIG. 1 is a flowchart of a method of estimating the effectiveness or safety of a medicine;

FIG. 2 is a schematic of a computer system operable to carry out the process shown in FIG. 1;

FIG. 3 is a more detailed schematic of the computer system of FIG. 2;

FIG. 4 is a schematic of the data flows in the computer system of FIG. 2;

FIG. 5 is a flowchart showing the process of FIG. 1 in more detail;

FIG. 6 is a schematic of an annotation system shown in FIG. 3;

FIG. 7 is a flowchart of a process carried out by the annotation system of FIG. 6 to improve classifier accuracy;

FIG. 8 is a flowchart of a corresponding process carried out by the computer system of FIG. 3 to improve classifier accuracy;

FIG. 9 is a screenshot of an annotation application running on the annotation system of FIG. 6;

FIG. 10 is a further screenshot of the annotation application running on the annotation system of FIG. 6;

FIG. 11 is a yet further screenshot of the annotation application running on the annotation system of FIG. 6;

FIG. 12 is another screenshot of the annotation application running on the annotation system of FIG. 6;

FIGS. 13A to 13F are graphs of feature indicators calculated for posts relating to specific medicines; and

FIG. 14 is a graph of a summary indicator of a patent confidence level in a specific medicine based on the feature indicators of FIGS. 13A to 13F.

### DETAILED DESCRIPTION OF AN EXAMPLE EMBODIMENT

An embodiment of a process and system for estimating the effectiveness or safety of a medicine will now be described.

In overview, the process involves analysing patient commentary related to medicines, in order to determine specific, objective and measurable indicators of a medicine's effectiveness or safety, which can be used to monitor or in some cases to improve the effectiveness and/or safety of that medicine. The process normally produces a summary estimate of confidence in the medicine, which estimate has a relatively direct correlation to effectiveness and/or safety, but can also provide additional statistical information to help identify specific areas of concern. The present process can reduce or eliminate the need for manual, human interventions with patients in order to obtain this type of information. The patient commentary is typically derived from a range of sources, including posts on websites, social media posts generally, and more structured sources, such as apps for monitoring medicine use. The process will now be described in more detail.

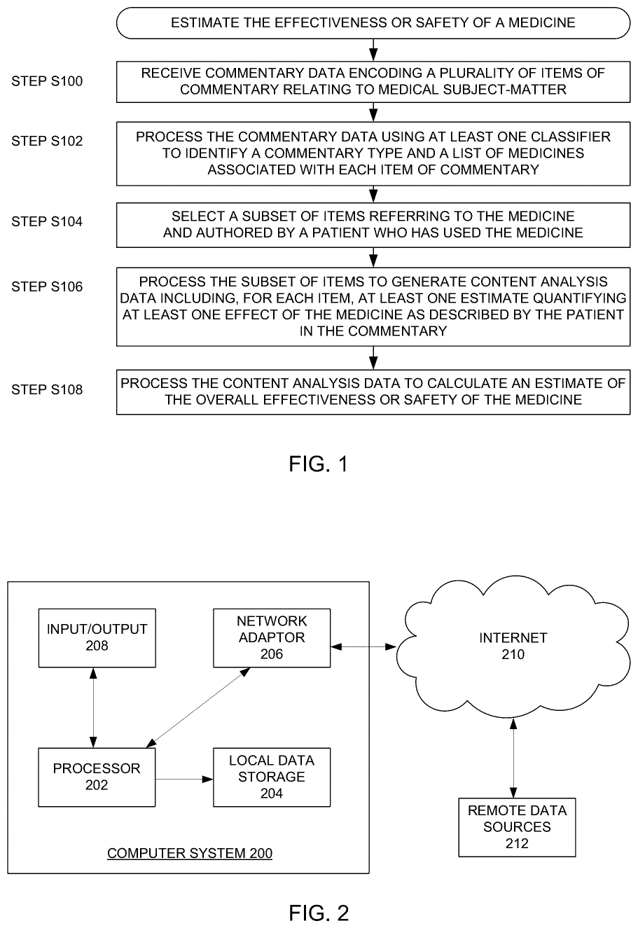

FIG. 1 is a flowchart of a method of estimating the effectiveness or safety of a specific medicine. In step S100, commentary data is received. The commentary data encodes a plurality of items of commentary (for example, social media posts such as Twitter® tweets, Reddit® forum posts, and so on). The data may be received and/or provided in any appropriate form, and may, for example, be converted from the original source into a standardized format, such as XML, CSV, HTML, and so on. The data may be processed and stored in a granular format, or may be treated as a single block of data. Typically the raw commentary data is selected from specific forums, or filtered using keywords, some combination of the two, or otherwise pre-filtered to return at least approximately relevant subject-matter in general.

In step S102, the commentary data is processed using at least one classifier to identify a commentary type and a list of medicines associated with each item of commentary. The commentary type typically distinguishes between different authors (patient and medical professional, for example) and between different types of commentary (personal experience, news items and factual information, for example).

In step S104, a subset of items is selected on the basis of whether each item of commentary refers to the specific medicine and is authored by a patient (that is, the commentary type is ‘patient voice’, as opposed to ‘medical professional voice’, ‘news report’, and so on).

In step S106, the subset of items of commentary is processed to generate content analysis data, which includes (for each item) at least one estimate quantifying at least one effect (or aspect of the effect) of the medicine as described by the patient in the commentary. The estimates are typically in the form of feature indicators corresponding to entity types identified by the NER classifier, as will be described in more detail below.

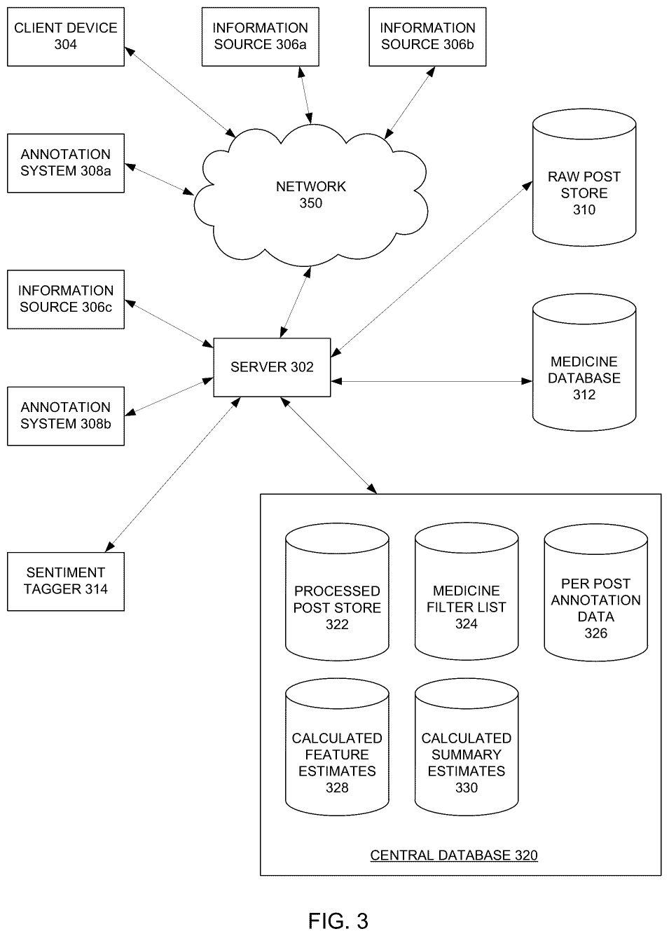

FIG. 2 is a schematic of a computer system operable to carry out the process shown in FIG. 1. In its simplest form, the computer system 200 includes at least one processor 202, local data storage 204 for computer program code, temporary data, and any other volatile or non-volatile storage needs of the or each processor. The system 200 also includes at least one network adaptor 206, and appropriate input/output means 208, such as a display, a keyboard and a pointing device, and the like. The computer system 200 may connected via the Internet 210 or any other appropriate network or local connection to various remote data sources 212 or to local data sources (not shown). The various aspects described above may, as appropriate, be distributed in any appropriate fashion, and need not all be provided in the same location.

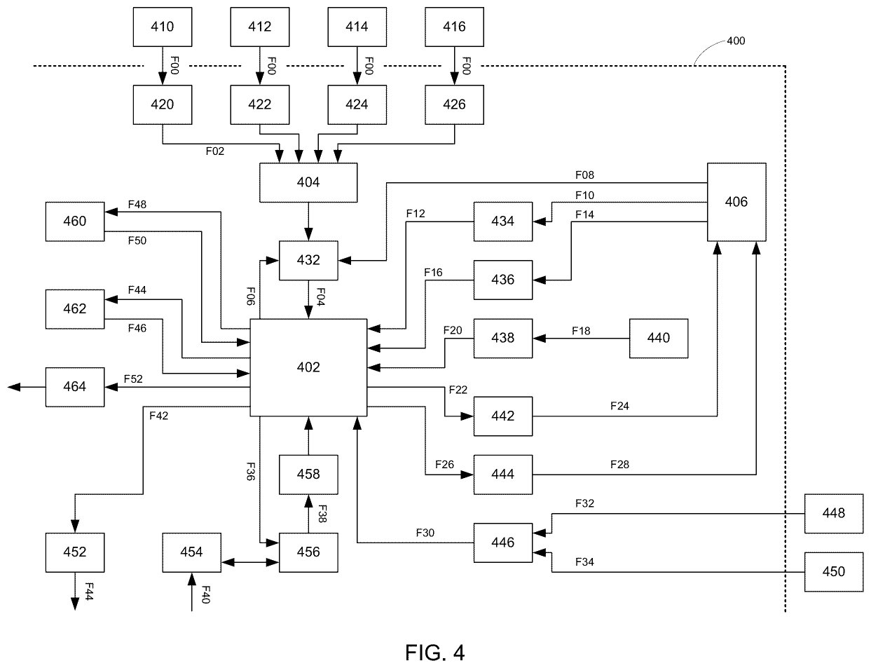

FIG. 3 is a more detailed schematic of the computer system of FIG. 2. A server 302 in this case essentially performs all of the functions of the computer system 200 shown in FIG. 2, but separate servers may be provided in respect of different aspects of the system's functionality, or any other appropriate architecture may be used. The server 302 may be accessed by one or more client devices 304, whether a customer device or otherwise. Like the client device 304, information sources 306a, 306b are accessible via a network 350 such as the Internet. It is possible also to have locally connected information sources 306c, for example providing data from an app associated with the provider of the server 302 and optionally running under the control of, or otherwise with the assistance of the server 302. Annotation clients 308a, 308b may also be provided to assist with training the aforementioned classifiers, either remotely (308a) or locally (308b). The server 302 has access to multiple databases, including the raw post store 310 for storing downloaded and/or normalized social media posts and other patient voice commentary data. The server 302 can also access a medicine database 312, which contains a list of brand names and generic names for medicines provided within particular jurisdictions (such as the UK and the US). A sentiment tagger 314 is also accessible for marking up individual posts as having a positive, negative or mixed sentiment. The tagger 314 may alternatively be provided within the system (for example on or via the server 302), and different sentiment scoring methods are of course possible. An alternative form of sentiment tagger 314 is also possible which marks up individual parts of a post separately (see below for more details). Typically a central repository 320 stores other discrete data sets such as a processed post store 322, a medicine filter list 324 with a list of keywords or other search terms for carrying out the initial filter on the downloaded posts (or, rather, before the posts are downloaded), per post annotation data 326 including the outputs generated by the classifiers for each post/item. Also provided in database 320 are the calculated feature estimates (feature indicators) 328, and the calculated summary estimates/indicators 330 which are produced for each medicine of interest (either as a regular task, or on demand, or both).

As regards the medicine database and medicine data, there are for example different ways to classify medicines. The World Health Organisation (WHO) Anatomic Therapeutic Chemical (ATC code) classifies medicines by therapeutic indication, whereas the FDA classifies medicines by mode of action. Other systems such as ICD-9, ICD-10 and SNOMED-CT also exist and can be used as and where appropriate. The choice of classification is preferably dependent on the selected jurisdiction or jurisdictions, but different classifications can be combined as and where appropriate also.

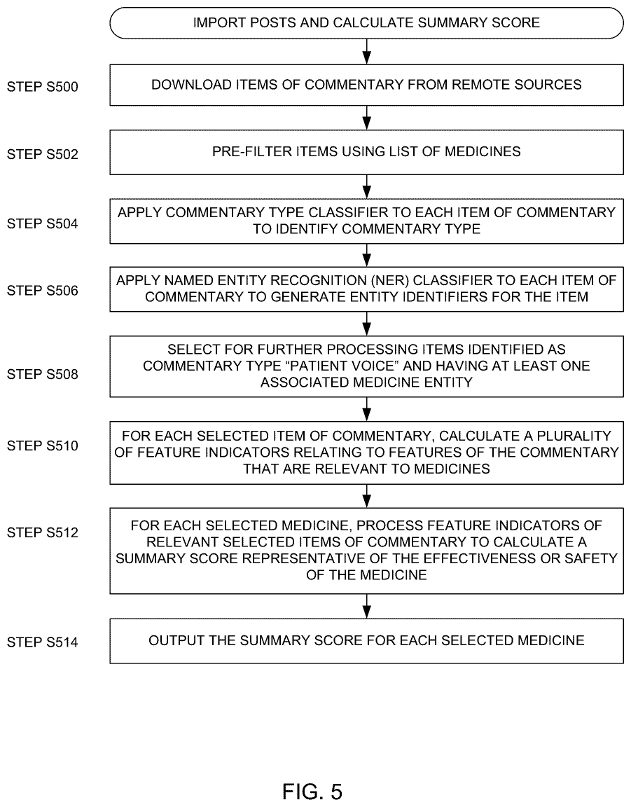

FIG. 4 is a schematic of the software architecture and data flows in the computer system of FIG. 2. Entities in the system are shown with normal numerals, and data flows are indicated with the prefix ‘F’.

The computer system 400 includes data sources 410, 412, 414, 416 which may, for example, include major social media websites and apps such as Reddit® and Twitter®, custom apps reporting anonymised medical commentary and/or structured data, and specialist forums frequented (at least) by patients. Raw downloaders 420, 422, 424, 426 handle the downloading of posts (F00) from sources 410, 412, 414, 416 respectively. The downloaders are customised to deal with a particular API and/or post format, and output (F02) the posts in a normalized format, tagged with raw data source specific data. For example, a Twitter® post downloader may store specific data relating to hashtags used, and numbers of likes and retweets.

Downloaded and normalised posts are stored in the raw post and normalised post store 404, which in turn outputs the normalised posts through the normalised post ingester (or importer) 432. The ingester 432 receives (F06) a list of the medicines to filter on, which may for example be altered dynamically depending on the area of medicine currently being analysed. The ingester 432 also receives (F08) data encoding the trained ‘patient voice’ classifier, trained NER model, and medicine gazetteer, from the model and gazetteer store 406. The ingester 432 processes the posts as described above, and outputs (F04) to the central database (402) the normalised posts that contain a medicine in the medicine filter list, are classified as being ‘patient voice’, and which contain at least one MEDICINE entity (see below).

The model/gazetteer store 406 provides (F10, F14) the patient voice classifier model data 434 and the NER classifier model data 436 to create and output (F12, F16) per-post patient voice classifications and per-post detected NER entities to the main database 402. An external language processing module 440 provides sentiment analysis results (F18) to a sentiment tagger 438, which in turn outputs (F20) a per-post sentiment measure to be stored in the central database 402. In the preferred embodiment, the Microsoft Cognitive Services system is used as the processing module 440, but any appropriate internal module may be provided instead or in addition. A medicines database 446 collates data (F32, F34) from medicine databases for various different countries, such as (for example) the UK medicines/MHRA data set and the US medicines/FDA data set 450, which are provided outside the system 400. The medicines filter list (which is typically built or adjusted depending on a particular data source or scope of enquiry) is provided F30 and stored in the database 402.

The filtered, normalised posts and the model outputs relating to each post are sent (F48) to a feature calculator module 460, which calculates various separate features indicators relating, for example, to brand count, averaged sentiment, MEDICINE entity count, SYMPTOM entity count, FEELING entity count, and so on. Per medicine feature indicators are returned (F50) to the database 402. The per-medicine features are then sent (F44) to a summary score module (typically a patient confidence score module) 462, and summary score for specific medicines are returned (F46) to the database 402. The summary score (patient confidence score, FCS) is output (F52) to an output module 464 along with any other appropriate or requested statistics such as brand count, trending entities, and so on.

Processed, downloaded patient voice posts and manually annotated posts are transmitted (F42) to a visualisation module 452 which presents (F44) data to annotator system users. The annotator users can submit (F40) adjustments to the annotation data via a web service 454 in communication with a local database 456. The database receives (F36) the posts in pre-annotated form by patient voice classifier and NER system. Manually annotated posts are submitted (F38) to an importer 458 which returns the adjusted annotation data to the central database 402. In the preferred embodiment, the Microsoft PowerBI module provides the visualisation 452, and the Doccano Text Annotation Platform is used to carry out the annotation 454, 456, in combination with a Doccano Importer 458. Multiple annotation systems may be provided for multiple annotators, with appropriate duplication of elements 452, 454, 456 and 458 where appropriate or necessary.

At an appropriate time after annotations have been made by the annotators and imported via the importer 458, manually classified posts for training (F22) and manually NER tagged posts for training (F26) are passed to the patient voice trainer 442 and NER model trainer 444, updates the model/gazetteer store 406 with the (re)trained versions of the models (F24, F28).

It will be appreciated that pre-filtering the posts for annotation greatly improves the efficiency of the annotation process, both by eliminating irrelevant items of undesirable commentary types (such as medical professional voice, or news items) and also by pre-populating the annotation data with suggested values output by the NER classifier. This in turn improves the quality of the data output by the classifiers, as a higher standard of annotation can more easily be provided, more quickly.

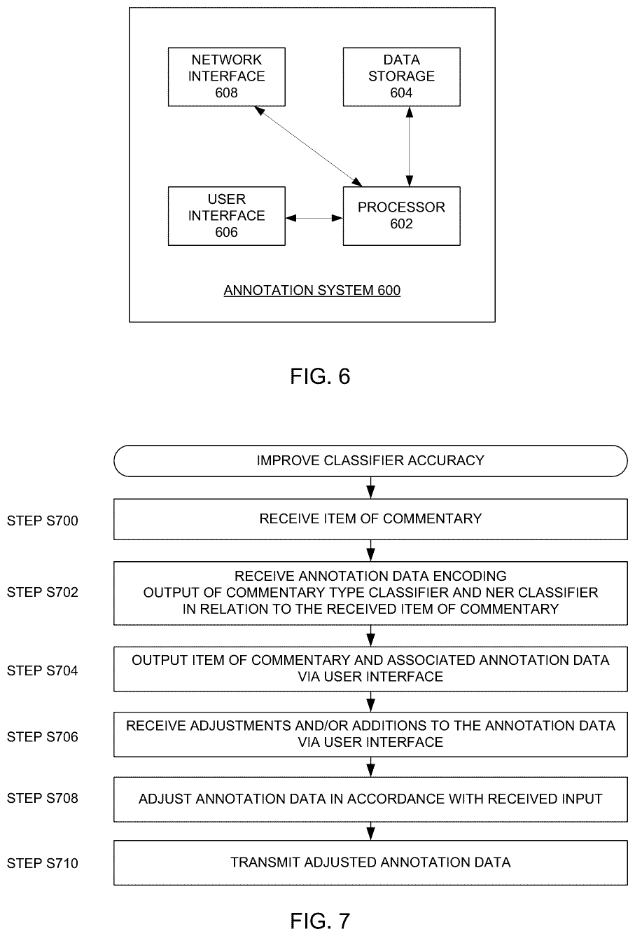

FIG. 5 is a flowchart showing the process of FIG. 1 in more detail, detailing the process of importing posts and calculating summary scores (again).

In step S500, items of commentary are downloaded from remote sources, as before. In step S502, the items are pre-filtered using the medicine filter list. In step S504, the commentary type classifier (patient voice classifier) is applied to each item of commentary to identify the commentary type (patient voice or other). In step S506, the named entity recognition (NER) classifier is applied to each item of commentary to generate entity tags/identifiers for each item. Typically steps S502, S504 and S506 are carried out in an appropriate order but substantially simultaneously by the post ingester shown at 432 in FIG. 4.

In step S508, items which are identified as being ‘patient voice’ type and which have at least one associated MEDICINE entity are selected for further processing. In step S510, for each item of commentary, a plurality of feature indicators (quantitative estimates) are calculated, relating to features of the commentary that are relevant to medicines. In step S512, for each selected medicine, the feature indicators are processed to calculate a summary score representative of the effectiveness or safety of a particular medicine. The summary score is then output at step S514, along with any other appropriate or requested information.

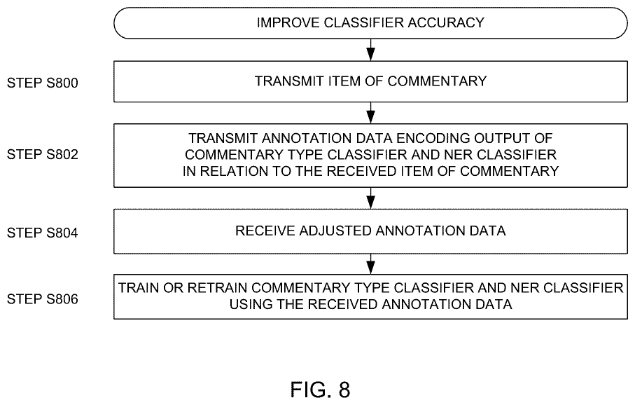

FIG. 6 is a schematic of an annotation system shown in FIG. 3. The system 600 includes at least one processor 602, local data storage 604 (for example for storing computer program code), a user interface 606 and a network interface 608.

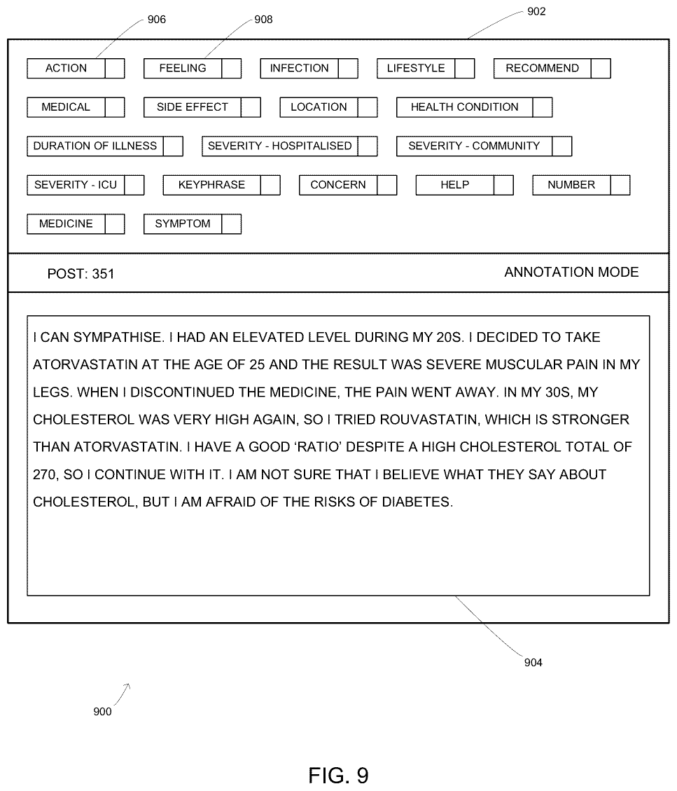

FIG. 7 is a flowchart of a process carried out by the annotation system of FIG. 6 to improve classifier accuracy. In step S700, an item of commentary (such as a social media post) is received for processing (typically in response to a request to retrieve it from an annotator). In step S702, annotation data is received which encodes the output that has been produced by the commentary type (patient voice) classifier and the NER classifier in relation to the received item of commentary. In step S704, the commentary item is output (at least in part), for example by displaying in a text box on screen (not shown), and so is the associated annotation data. Doccano is used in the preferred embodiment, and highlights annotated phrases with an appropriate colour indicating the entity type. Other approaches are of course possible. In step S706, the annotator can provide adjustments and/or additions to the annotation data via a user interface (such as mouse and keyboard, for example), for example by adding annotation(s) to the commentary, or removing annotations. In step S708, the annotation data is then adjusted appropriately, and in step S710, the modified annotation data is then submitted back to the central database for further processing (for example to retrain the classifiers with the new data). It will be appreciated that manual annotation is used because of the high quality of data and consequently high quality of training that it provides, but automated machine learning processes are also possible, instead of, or as well as, manual annotation. The annotation systems can be used either to annotate ‘fresh’ posts or to perform quality control operations on previously annotated posts. Normally, new posts on the system are pre-annotated using the initial output of the NER and patient voice classifiers, but it is of course possible to begin annotation from scratch, if preferred.

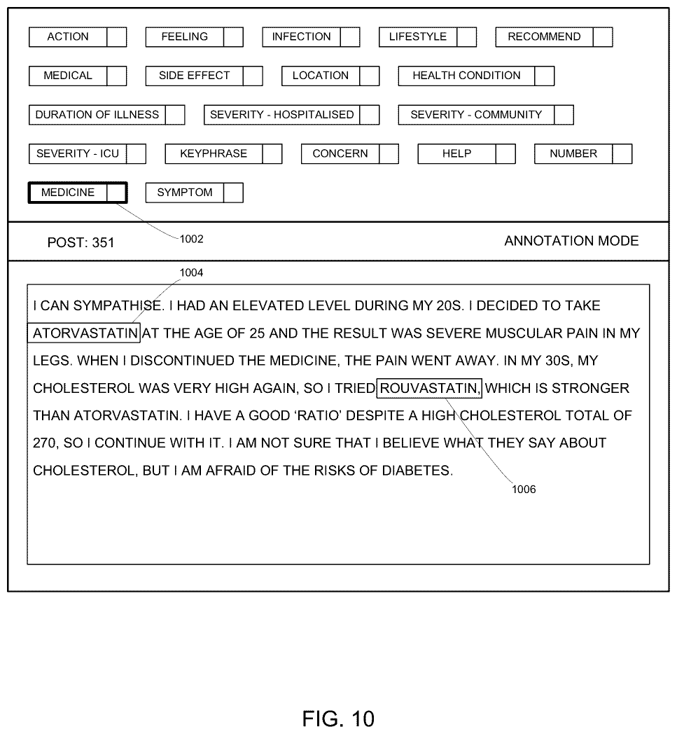

FIG. 8 is a flowchart of a corresponding process carried out by the computer system of FIG. 3 to improve classifier accuracy. In step S800, an item of commentary is transmitted to the annotation system, typically in response to a request for the item. In step S802, the associated annotation data, encoding the output of the post classifiers, is transmitted also (typically in a single transmission for both data items). In step S804, the modified annotation data is received from the annotation system. In step S806 (which may occur immediately after step S804, but may alternatively or additionally occur on a scheduled basis, on demand, or in response to receiving a certain number of modifications, for example), the modified annotation data is used to retrain the classifiers.

The processes and systems mentioned above will now be described in further detail.

It will be appreciated that further enhancements may be made to the classifiers, for example to merge them into a single classifier. The use of the NER classifier as it is provides a significant advantage over simple keyword searching. Keyword search is not able to detect context, for example.

By way of example, medicine names such as ‘Heather’, ‘Lyme’, ‘Nikki’, ‘Muse’, and so on are difficult to identify by any means, but the NER model is able to recognise the medicine name correctly through the recognition of language and context.

For example, a false positive for ‘Heather’, which the NER classifier may correct reject, would be in a sentence such as:

“Heather was really good forme. She actually took me to the cinema loads and helped me through things.” (Heather is a person, not a medicine)

However, a true positive for ‘Heather’, which would be identified correctly by the NER classifier, would be in a sentence such as:

“So I've only been taking Fakemed for a week Previously, I was taking Heather for about six months” (Heather is a medicine here)

Similarly, a false positive for ‘Nikki’, which would normally be rejected by the NER classifier (if well trained) would be in a sentence such as:

“Nikki, I'm very glad you're getting some relief. ”(Nikki is a person, not a medicine)

And a true positive would be along the following lines:

“Question about Nikki (Yax generic) . . . should I switch? I was taking the Fakemed generic for six months, and nowt am on the Nikki generic.” (Nikki is a medicine here)

Again, it will be evident that a simple keyword search (or indeed a non-simple keyword search) would not be able to distinguish between these two examples. But a keyword search in conjunction with the classifier would result in good accuracy with greater efficiency due to the reduced amount of data to process.

One important (yet optional) aspect of the preferred embodiment is to provide separate and essentially independent classifications of posts according to medicine or treatment area. The exact granularity of subject-matter is a question of judgement, client demand, and trial and error. It was found that the ‘patient voice’ differed according to medical subject-matter, to the extent that NER and patient voice classifiers trained on all posts were typically outperformed by classifiers trained on specific medical/medicine subject-matter areas.

Two example areas of medicine (domains') which were studied in some detail were heart medicine and skin medicine. Separate and diverse sets of data sources, keywords to filter results with, and annotation data sets were provided for each as appropriate. Both the patient voice and NER classifiers can be selected in dependence on a medicine area (or symptoms, or any other property associated with specific medicines) for best results, or at least one of the classifiers (preferably the NER classifier) can be.

The following table includes examples of ‘sub-reddits’ (individual forums within the Reddit website) for the two domains of interest in the present exemplary embodiment:

TABLE 1
Sources of patient commentary from the Reddit website

| Domain | Subreddit |
|---|---|
| cardiovascular | askCardiology |
| cardiovascular | Cardiomyopathy |
| cardiovascular | CHDs |
| cardiovascular | Cholesterol |
| cardiovascular | HeartAttack |
| cardiovascular | HeartDisease |
| cardiovascular | hypertension |
| cardiovascular | ihadastroke |
| cardiovascular | stroke |
| skin | Accutane |
| skin | AusSkincare |
| skin | Dermatology |
| skin | Eczema |
| skin | EczemaCures |
| skin | Psoriasis |
| skin | popping |
| skin | skin |
| skin | SkincareAddicts |
| skin | teenagers |

Typical search terms used to pre-filter the results from these forums are shown in the following table:

TABLE 2
keywords for filtering downloads from Reddit website

| Domain | Search term |
|---|---|
| cardiovascular | Accupril |
| cardiovascular | ACE inhibitor |
| cardiovascular | blood clot |
| cardiovascular | beta blocker |
| cardiovascular | cardiac arrest |
| cardiovascular | cardiac infarction |
| cardiovascular | edoxaban |
| cardiovascular | fast heart rate |
| cardiovascular | high blood pressure |
| skin | balneum |
| skin | dermatitis |
| skin | diprobase |
| skin | eczema |
| skin | flakey skin |
| skin | itchy skin |
| skin | psoriasis |
| skin | skilarence |
| skin | sore skin |

It will be appreciated that search terms include not only specific medicines (brand names and generic names), but may also include symptoms, side effects, types of medicine, treatment types, and so on.

The following table gives examples of the NER classifier outputs, along with related statistics such as sentiment summary and the brand count in a post (number of times a medicine is mentioned) which are later used to produce feature indicators.

TABLE 3
Classifier outputs and statistics for items of commentary

| | | Sentiment | Brand count |
|---|---|---|---|
| Entities | Sentiment | summary | on post |
| MEDICINE - Fakemed | positive - 0.4 | mixed | 3 |
| FEELING - terrified | neutral - 0.0 | | |
| FEELING - scared | negative - 0.6 | | |
| FEELING - embarrassed | | | |
| MEDICINE - Fakemed | | | |
| MEDICINE - Fakemed | positive - 0.0 | negative | 3 |
| SYMPTOM - cold sores | neutral - 0.02 | | |
| SYMPTOM - cold sores | negative - 0.98 | | |
| SYMPTOM - cold sores | positive - 0.01 | neutral | 1 |
| MEDICINE - Fakemed | neutral - 0.98 | | |
| | negative - 0.01 | | |
| MEDICINE - Fakemed | positive - 0.5 | mixed | 1 |
| SYMPTOM - itchy eyes | neutral - 0.0 | | |
| SYMPTOM - dry skin on | negative - 0.5 | | |
| my face | | | |
| MEDICINE - steroid | positive - 0.52 | mixed | 1 |
| SYMPTOM - skin got | neutral - 0.0 | | |
| really tight and dry | negative - 0.48 | | |
| SYMPTOM - red | | | |
| SYMPTOM - dry peeling | | | |
| skin | | | |
| MEDICINE - Fakemed | | | |

The first row in Table 3 corresponds to an item of commentary such as the following extract from a social media post (such as a Reddit® post):

“I want to talk about Fakemed, because I used to be terrified about those symptoms. I was scared and embarrassed. Oh it was terrible. But I used Fakemed for a while and things are much better now”

The second row of Table 3 corresponds to an item of commentary such as the following:

“You want to use Fakemed for your cold sores? No way. I tried it on my cold sores and the result was terrible.”

And similarly for the remaining rows.

In a further embodiment, multiple sentiments are analysed, on an appropriate conceptual axis. For example, anger (caused as an effect of a medicine) can be categorised on an axis between calm and angry. Likewise neuroticism on an axis between grounded and neurotic. Happiness can be measured on an axis between unhappy and happy, and so on. Similar techniques to those described above in relation to Table 3 can be used in these cases. By tracking the multiple sentiments and optionally drawing correlations between them (relative to each other and/or relative to other measured properties of the commentary), further insights can be gained.

The annotation will briefly be described again. The following description illustrates how the Doccano system can be used in the context of the present embodiment to create the annotation data.

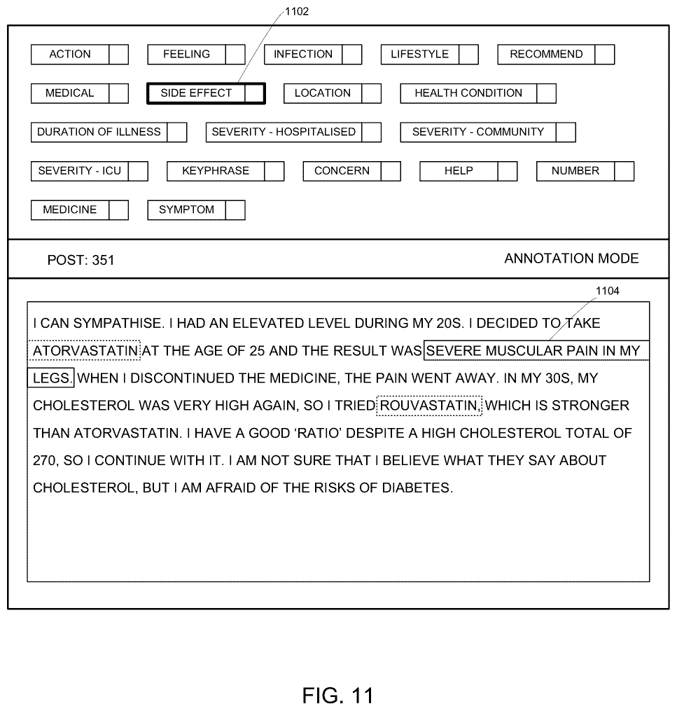

FIG. 9 is a screenshot of an annotation application running on the annotation system of FIG. 6. The screenshot 900 includes a tag window 902 and a commentary window 904. A number of tags 906, 908 and so on are shown. The user of the annotator system is able to select an area of text in the commentary window 904 and then to select a tag 906, 908 to add an annotation to the commentary of the appropriate entity type (such as ACTION, FEELING, INFECTION, and so on). The commentary example is synthetic (that is, not a real example).

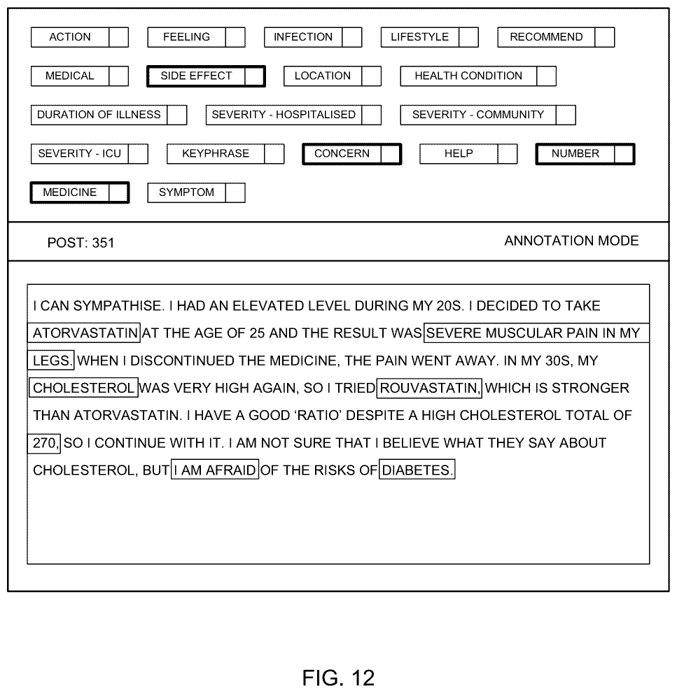

FIG. 10 is a further screenshot of the annotation application running on the annotation system of FIG. 6. Here, the operator chooses to add a couple of tags for the MEDICINE entity 1002. The first example of Atorvastatin 1004 and Rouvastatin are tagged. Subsequent occurrences of the same tag need not be unless worded differently.

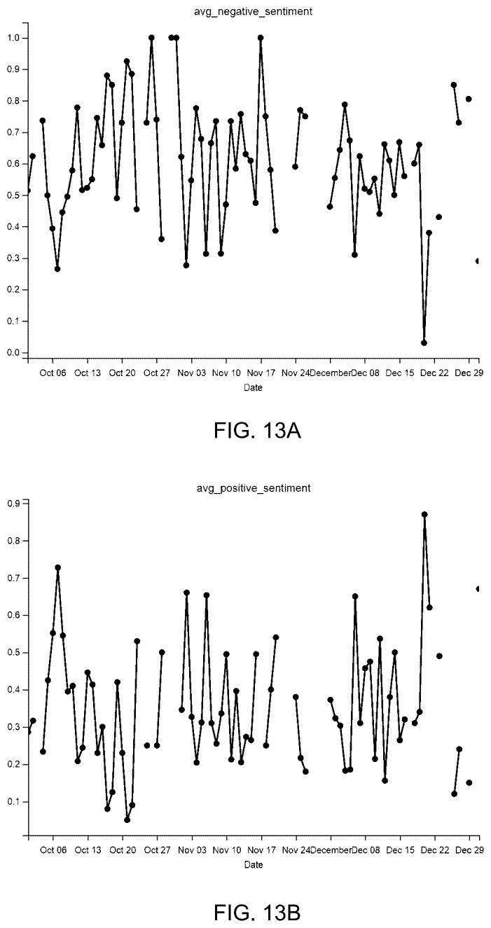

FIG. 11 is a yet further screenshot of the annotation application running on the annotation system of FIG. 6. Here the side effect tag 1102 is applied to the text 1104 relating to a severe muscular pain in the patient's legs. Other relevant tags are added in a similar way until the text is fully annotated. The tags that have been applied will help the NER classifier to produce more accurate classifications.

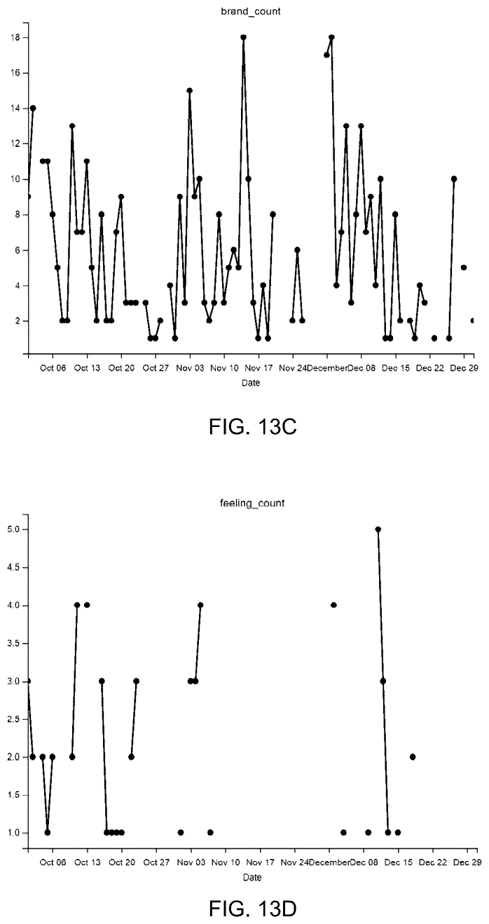

FIG. 12 is another screenshot of the annotation application running on the annotation system of FIG. 6. Here we see all of the tags now added. Various tags are used in various ways to contribute to the determination of the effectiveness or safety of the medicine. The tags which define an effect of the medicine on the patient are of greatest interest, and contribute the most to the ultimate summary score (such as the patient confidence score, for example). However, if no relevant tags are provided, the sentiment estimated for the post (positive, negative or neutral) still contributes to the overall score.

The quantitative estimates of effects of the of a medicine (that is, the feature indicators) will now be described in more detail.

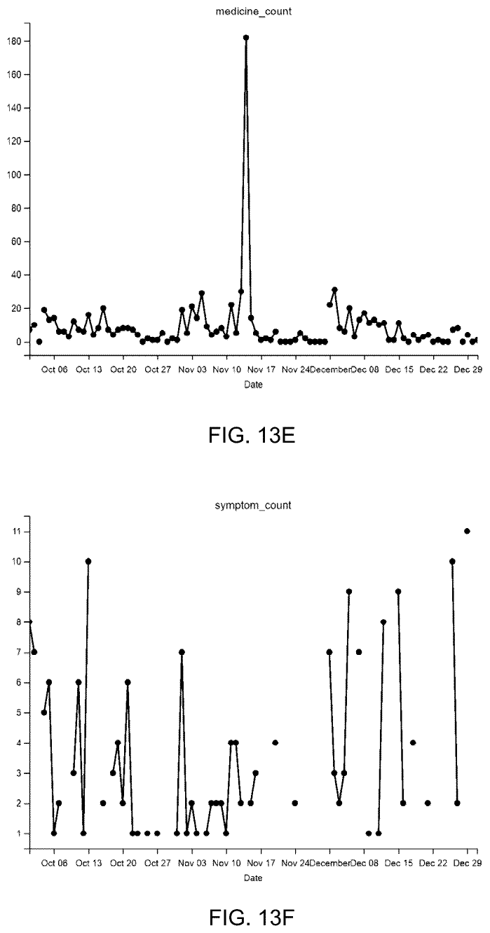

FIGS. 13A to 13F are graphs of feature indicators calculated for posts relating to specific medicines, and plot aggregate/average feature indicators for a specific medicine for specific time periods. FIGS. 13A and 13B show the average negative and positive sentiments for all posts relating to a particular medicine, varying over time. In some cases, no relevant data is found for a particular day (or other time period), and a gap in the graph occurs. FIG. 13C shows the aggregate brand count for a particular medicine for a particular day, again showing many gaps. FIG. 13D shows the aggregate count of feelings per day. FIG. 13E shows the aggregate count of MEDICINE entities per day, and FIG. 13F shows the aggregate count of SYMPTOM entities per day, as output by the NER classifier. It will be noted that all of the graphs 13A to 13F show high variance and partially incomplete data. It is objectively difficult to determine any underlying trends from the graphs in FIGS. 13A to 13F.

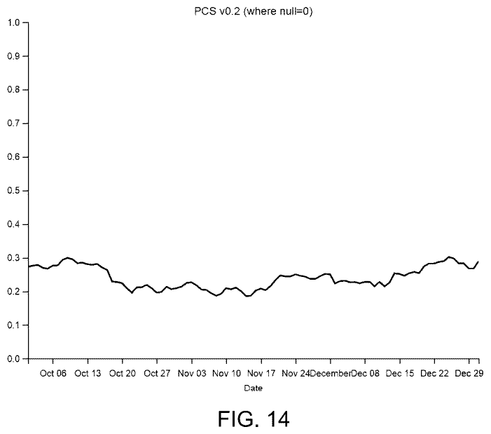

FIG. 14 is a graph of a summary indicator of a patent confidence level in a specific medicine based on the feature indicators of FIGS. 13A to 13F. The result is much clearer, and is much easier to read and to use. There is a continuous and constrained movement in the summary value over time.

The summary indicator, as mentioned above, is known as a Patient Confidence Score (PCS). The feature indicators used to create the PCS are selected to highlight the confidence aspect (emphasising the FEELING entity). Different indicators could be prioritised to focus especially on the effectiveness or especially on the safety of the medicine, though the exact mix depends on the area of medicine, the nature and severity of typical symptoms and side effects, and so on. Different indicators may have other names as appropriate. The PCS is one specific example.

Any appropriate combination of the feature indicators is possible. In the present case, the summary value is scaled to a range within 0 and 1, where 0 represents a totally negative sentiment from patients towards the medicine, and 1 represents a totally positive sentiment. To create the PCS, the brand count, MEDICINE entity count, SYMPTOM entity count and FEELING entity count are normalised and combined to determine a relevance contribution, in turn determining the relative contribution each post will make to the summary score (PCS). A simple moving average of the positive and negative sentiment feature indicators is scaled in accordance with the relevance contribution in order to determine the overall summary score. As noted, the precise mix of entities considered and their relative weighting is something best determined by trial and error after an appropriate selection of data sources and feature indicators has been finalised.

Once the PCS score is calculated for a range of time periods for a particular medicine, it can be output in any appropriate fashion, for example with additional statistical information. There may be a further step of annotation (manually or otherwise) of key events on the timeline of the summary score (PCS), for example to attempt to explain the cause of significant changes in the summary score. For example, changes in the price of a medicine could be automatically determined and annotated on the timeline, or otherwise. An output showing the results for multiple medicines, for example a plurality of medicines produced by the same pharmaceutical company, may be provided.

One additional form of output is a list of entity trends, such as shown below in Table 4.

TABLE 4
example entity trends

| Entity | Count | Change |
|---|---|---|
| Fakemed | 21 | n/a |
| Shortness of Breath | 6 | 3 (+0.5%) |
| Ultrasound | 5 | n/a |
| Anxious | 4 | 1 (+0.3%) |
| Blood Thinners | 4 | n/a |
| Dvt | 3 | −4 (−1.3%) |

A method of calculating the Patient Confidence Score (PCS) will now be described in more detail.

Each post can be defined as having a positive sentiment and/or a negative sentiment. Posts can also be classified as having a neutral sentiment, and these posts are discarded. Three variables are defined as follows:

Smoothed Mean Positive Sentiment=Total Positive Sentiment/Total Posts (1)

Smoothed Mean Negative Sentiment=Total Negative Sentiment/Total Posts (2)

Normalised Sentiment=Smoothed Mean Positive Sentiment/(Smoothed Mean Positive Sentiment+Smoothed Mean Negative Sentiment) (3)

A relevance weighting is also computed, in dependence on four different counts derived from the stored data. The four counts and their weighting is as follows:
Brand Count (0.7): a total mention count for the medicine of the corresponding PCS on a given day (such as “Fakemed”).
Medicine Count (0.1): a total mention count for any different medicine to the Brand mentioned above.
Symptom Count (0.1): a total mention count for any symptoms on a given day (such as “sweating”, “mild dizziness”, “poor memory”).
Feeling Count (0.1): a total mention count for any feelings on a given day (such as “confident”, “anxious”, “embarrassed”).

Other weightings can be chosen as desired, for example to give equal weighting to all counts. The weighting is calculated by taking the mean of the four different counts above, over the same range of time as the Normalised Sentiment (see below), normalising these counts to a range of (0, 1), applying the specified weighting to the four different normalised counts, and then multiplying by a scaling factor (typically 0.2) to calculate a Relevance Weighting (otherwise known as a Contextual Feature Score) for incorporation in the final PCS score.

In this embodiment, the Normalised Sentiment is clamped to values of (0.1, 0.9), or any other appropriate sub-range within the full range (0, 1), such as (0.2, 0.8), (0.3, 0.7), and so on, so that its contribution to the overall PCS score is limited to a desired degree. This can lessen the chances of lots of negative or positive posts on any one particular day affecting the score adversely. Because the sentiment value is not fully weighted, additional weighting is given to the contextual features mentioned above. This means that in order to achieve a PCS of over (say) 0.9 or lower than (say) 0.1, depending on the clamping done, contextual features must be present in the post. Without these, no matter how many voices and how negative/positive these voices are, the score cannot reach its maximum (1) or minimum (0).

In a simple example, the PCS is calculated using a conditional formula:

Normalised Sentiment>=0.5: PCS=Normalised Sentiment+Relevance Weighting;

Normalised Sentiment<0.5: PCS=Normalised Sentiment−Relevance Weighting. (4)

This formula experiences discontinuities as the Normalised Sentiment tracks across the 0.5 value, however, so it is preferable to replace the conditional outcome (depending on whether the observed sentiment is above or below 0.5) with an interaction variable that depends directly on the sentiment.

This revised method is broken down into two parts: firstly, a standardised version of normalised sentiment is calculated. This is a transformation converting the previous sentiment score of between 0 and 1 into a standardised sentiment of between −1 and 1. For example, if the normalised sentiment is 0.5, the standardised sentiment is 0, or if the normalised sentiment is 0.25, the standardised sentiment would be −0.5.

Secondly, the standardised sentiment is multiplied by the contextual weighting (thereby the standardised sentiment is the ‘interaction variable’), providing an impact from changes to various of the count variables derived from the model output (such as medicine counts, feeling count, and symptom count) in the PCS. Because the interaction variable (standardised sentiment) will be around 0 when the normalised sentiment is 0.5 (that is, when the sentiment is mixed, being neither positive nor negative overall), the contextual weighting has little effect on the PCS. But this can provide improved results relative to the original method, where the PCS can swing suddenly around 0.5 without any obvious cause. In the present method, if the sentiment for a given medicine tracks close to 0.5 for a long period of time, then a large level of variation in the overall PCS is less likely to be seen.

The posts used to calculated the Smoothed Mean Positive Sentiment and the Smoothed Mean Negative Sentiment are taken from the last 365 days of data (or thereabouts, 366 days for a leap year, and so on). This avoids having to wait for future data or having any future events causing a change in the normalisation, for example as is the case when using a moving window centred on the present day. This choice of window also reduces the effect of seasonal variations, and creates relatively consistently normalisation and avoids excessive ‘spikes’ in the output. It will be appreciated that some conditions depend on seasonal factors such as average temperature, humidity and sunshine, whether directly or as a consequence of the impact of these factors on the environment or organisms within the environment. At least the use of medicines, if not their efficacy for similar reasons, may therefore vary by season. Accordingly, an appropriate choice of data window can help to eliminate such variations.

The method also distinguishes between different types of missing data. When there are days when no feelings or safety factors were mentioned in posts for a particular medicine, a value of 0 is used to score the number of posts. Where posts are unavailable for a particular day, however, due to problems with the data sources, then null data is used instead. This can help to reduce the impact of interruptions to data sources on the output metrics.

In more detail, if a number of contributions to the Relevance Weighting are zero, they will be included. However, if all contributions to the Relevance Weighting are zero, this will be interpreted (in the absence of any other information) as a failure to collect data. In this case, the PCS score will only be calculated using the Normalised Sentiment. Thus the overall score will not be ‘pulled down’ by an apparent low Relevance Weighting that is in fact an unknown Relevance Weighting due to insufficient data. Other methods may be used as appropriate to determine the validity of either the Relevance Weighting or Normalised Sentiment, and to make appropriate adjustments to the calculation of the PCS.

In addition to the steps above, additional ‘post curation’ filtering is provided to prevent posts being processed by the PCS based on social media site category (such as ‘subreddit’), author, and specific clauses found in the text of the post. This can help to ensure that posts created by bots, as well as irrelevant posts which get past the patient voice classifier, are filtered out before the PCS is calculated. With reference to the system diagram in FIG. 4, the post curation filter may be located within the post ingester 432, before or after the post ingester 432, or elsewhere within the system in communication with the central data store 402.

An example of a structured data source will now be described.

A medicine and pill reminder app provides post-prescription patient support. It provides general information about post-prescription medication for patients to view, and can be used by patients or carers of patients. It offers features including: smart reminders, a medicine log, multiple profiles, medicine ingredients, and a guide to how to use medicines. The app can also facilitate smart surveys, for example via social media websites.

It was discovered that aggregated data received from apps for managing medicines in various ways, and the like, was more community-based and mainstream, whereas data obtained via APIs from social media apps and websites were orientated more towards clinical use and discussion. It was found that social media posts generally provide more insight as regards recently-diagnosed patients who were perhaps struggling to get diagnosed, and were perhaps in shock about their new diagnosis, and had questions regarding what all the various medicines were, and so on. Both sources have their use.

In one variant of the present embodiment, an appropriate user interface is provided to allow multiple confidence scores to be produced, divided by cohort, or by data source. Types of filtering (including selecting different cohort types) are user selectable. The resulting summary scores can then be compared, as well as other outputs, such as most common identified entities. Appropriate modifications are made to the raw post processor and other parts of the system as necessary to facilitate this feature. Further divisions and subdivisions of data, and appropriate comparisons of resulting summary scores (of any type), are of course possible.

In one example, the summary scores may be broken down by age range, either numerical (0-20, 21-30, 31-40, and so on), or in discrete ranges (young, old), or qualitatively (patients on medicine X vs patients on medicine Y, or patients with symptom A vs patients with symptom B, and so on). The lists of identified entities can be compared for each cohort, for example, to identify entities that appear to be associated disproportionately with particular cohorts, and so on.

As mentioned above, typically one or more different types of classifications of medicine are used. The analysis can furthermore be divided not just be specific medicine but also by class of medicine, mode of action, therapeutic indication, and any appropriate way to divide the various forms of medicine data in the medicine database (or otherwise/elsewhere).

Data may be retrieved from the app in an unidentifiable and aggregated manner, preserving the confidentiality of patients and users of the app. Data can include details of medicines, reasons for taking a medicine, patient feelings, and related or unrelated medical conditions. This structured data can be used (fully anonymously) to provide what is essentially an item of commentary, albeit structured in origin. The data can be processed in any appropriate way as described above.

In terms of implementing the processes and systems described above, the Azure framework was used, in conjunction with the Microsoft® PowerBI and Cognitive Services products, and the Doccano annotation platform. With reference to FIG. 4, the raw post and normalised post store 404 and the model/gazetteer store 406 were implemented as containers in Azure Blob storage. Blocks 420, 422, 424, 426, 432, 434, 436, 438, 442, 444, 454, 458, 460, 462 were implemented as running a container in Azure Container Instances. The main database 402 and the medicines database 446 were implemented as an Azure database. Other configurations and platforms are of course possible.

As regards the classifier models mentioned above, the ‘spaCy’ off-the-shelf software was used for the models.

The patient voice classifier (which technically classifies multiple voices, of which patient voice is one) is a text categorisation model. It takes a social media post (the text) as input and outputs a label for that post. This label is one of the following classes:
Patient voice
Professional voice
News
Not relevant

Other categorisations (including more or fewer categories) are of course possible. The patient voice classifer (“PV classifier”) is trained on ‘gold standard’ annotated data using the TextCategorizer model from the Python software spaCy (at the spacy.io website). Specifically, spaCy version 2.3.2 was used.

When training the PV classifier and using it to label posts, the default configuration for spaCy's TextCategorizer model was used, namely the “ensemble” architecture. As per the spacy documentation (accessed via path api/textcategorizer #architectures on the spacy.io website), the ensemble architecture is a stacked ensemble of a bag-of-words model and a neural network model. The neural network uses a CNN with mean pooling and attention.

An n-gram size to use for the bag-of-words model that is part of the ensemble can be specified. The present embodiment uses an n-gram of 1.

The NER model is a named entity recognition model. It takes a social media post (the text) as input and outputs zero or more “entities” that are present in the text. Examples of the types of entities it can label words in the text with are:
MEDICINE
FEELING
SYMPTOM
Others cover disease, severity, persona, lifestyle etc.

The NER model is trained on ‘gold standard’ annotated data using the EntityRecognizer model from the Python software spaCy (at the spacy.io website), and specifically spaCy version 2.3.2.

When training the NER model and using it to tag posts with entities, the default configuration for spaCy's EntityRecognizer model was used. The model is backed by a neural network that uses a stacked embedding architecture. The details of spaCy's default EntityRecognizer are outlined in their video documentation (accessed via path watch?v=sqDHBH9IjRU on the www.youtube.com website).

In the preceding description, each post is processed as a single entity, in terms of having scores or properties assigned to it, and so on. In a variant of the above embodiment(s), each post may selectively be divided into a plurality of parts for classification, and each part of each post may be classified, processed and/or assigned attributes independently of any other part. Each part may correspond to one or more sentences.

For example, a post may contain two or more sentences, which may be treated as two or more respective parts. In one example, one part may contain a reference to a relevant medicine, symptom, and so on. Other sentences may not have any relevant references. In this case, only the apparently relevant part/sentence may be selected and processed further, and other parts may be discarded. This can improve the quality of information that is extracted, and reduce the amount of irrelevant information that is extracted.

In another example, a first sentence/part may express a positive sentiment about one medicine, symptom, and so on, and a second sentence may express a negative sentiment about another medicine, symptom, and so on. By dividing the post into separate parts, more useful information may be captured. Furthermore, the most useful information was found to relate to positive or negative sentiments. The combination of both positive and negative sentiments in a post that is treated as a single unit can result in an overall mixed or neutral sentiment, which would cause the entire post to be (wrongly) discarded.

In the above examples, each part is treated independently. From this, more information can be determined, and more granular data can be provided, allowing for more sophisticated information to be determined regarding medicine safety and so on.

However, it is possible also to combine parts so as to make inferences or otherwise derive additional information from their interrelationship. For example, a clear reference to a medicine in one part may allow ambiguities to be resolved in the second part of the same post, which might otherwise be discarded if considered in isolation.

To take an example of the above principles, we consider a post such as: “3 yr old has dilated cardiomyopathy. We went to Bigtown Children's Hospital and my son had a lower blood pressure than normal He is on multiple medications to treat his disease. Recently he started Newmed, which is amazing”

Analyzed as a whole, this post was given a score of 50% positive, 7% neutral and 43% negative, resulting in a classification of “Mixed”. However, considered at the sentence level, the last sentence which mentions the Newmed medicine is classified as having a 100% positive sentiment. Thus, by using the sentiment score for the sentence within the post that relates more specifically to the mention of the medicine, it is possible more accurately to represent the feelings towards the medicine brand.

Further refinements of the models and configurations thereof are of course possible, having due regard to the specific data sets which are processed and the nature of the specific medical area in question.

The present embodiment deals exclusively with text-based data sources, but it will be appreciated that text transcriptions of audio and/or visual sources can also be processed, and audio and/or visual sources can be processed directly also with appropriate adjustment of the embodiment described above. Classifiers can be created which act directly on audio-visual inputs, for example to provide effectively a speech-to-text functionality, or to classify the content more directly/holistically. References to social media posts and the like may connote socially sourced public opinions in general.

Although the present invention has been described above with reference to specific embodiments, it will be apparent to a skilled person in the art that modifications lie within the spirit and scope of the present invention.

1. A method of estimating the effectiveness or safety of a medicine, the method comprising:
receiving commentary data encoding a plurality of items of commentary substantially related to medical subject-matter;
processing the commentary data using at least one classifier to identify for each item a commentary type and a list of medicines associated with the commentary;
selecting a subset of items, from the plurality of items of commentary, identified as referencing the medicine and whose commentary type has been identified as commentary from a patient who has used the medicine;
processing the subset of items to generate content analysis data including, for each item, at least one estimate quantifying a respective at least one aspect of an effect of the medicine as described by the patient in the commentary; and
processing the content analysis data to calculate an estimate indicative of the overall effectiveness or safety of the medicine.

2. A method according to claim 1, wherein said at least one classifier includes a Named Entity Recognition, NER, classifier for identifying at least the list of medicines associated with the commentary.

3. A method according to claim 2, wherein the NER classifier additionally identifies references to at least one of: type of medicine, ailment, condition, symptom, potential side effect, possible medical outcome, and treatment type.

4. A method according to claim 1, wherein at least one said classifier further identifies personal experiences or feelings described by the patient.

5. A method according to claim 4, wherein processing the subset of items to generate content analysis data further comprises processing the personal experiences or feelings described by the patient.

6. A method according to claim 1, wherein said at least one aspect of an effect of the medicine includes at least one of: a perceived effectiveness of the medicine, a perceived safety of the medicine, happiness or unhappiness associated with the medicine, and satisfaction or dissatisfaction associated with the medicine.

7. A method according to claim 1, wherein processing the subset of items to generate content analysis data further comprises processing the subset of items to estimate a degree of positivity or negativity expressed by the patient in relation to at least one of: the commentary considered as a whole, each mention of the medicine individually, and every mention of the medicine considered as a whole.

8. A method according to claim 1, wherein processing the subset of items to generate content analysis data preferably further comprises processing the subset of items to calculate a sentiment estimate, encoding a measure of at least one of: positivity, negativity and neutrality, expressed by the patient in relation to at least one of: the commentary considered as a whole, each mention of the medicine individually, and every mention of the medicine considered as a whole.

9. A method according to claim 8, further comprising dividing each item into at least one part, and wherein processing the subset of items to calculate a sentiment estimate comprises processing each part separately and wherein each sentiment estimate relates to a respective part.

10. A method according to claim 8, further comprising processing each part to determine whether or not to exclude the part from further processing.

11. A method according to claim 1, further comprising processing each of the subset of items to identify at least one specific characteristic in the item and, if a said specific characteristic is identified, to exclude the item from further processing.

12. A method according to claim 1, wherein said at least one classifier includes a commentary type classifier.

13. A method according to claim 12, wherein the commentary type classifier is configured to identify at least one commentary type, selected from at least one of: patient opinion, medical reference data, medical professional opinion, scientific report, industry report, news report, and structured feedback.

14. A method according to claim 12, wherein the commentary type classifier is configured to identify at least one author type, selected from at least one of: patient, medical professional, medicine representative, research scientist, journalist; and other type.

15. A method according to claim 1, wherein at least one said at least one classifier is selected in dependence on the medicine.

16. A method according to claim 1, wherein selecting the subset of items further comprises filtering the subset of items by one of a plurality of cohorts, and calculating a respective estimate indicative of the overall effectiveness or safety of the medicine for each cohort.

17. A method according to claim 1, further comprising:
accessing a medicine database containing medicine data that encodes a plurality of medicine names associated with at least one jurisdiction;
retrieving data from the medicine database in accordance with a search query, the retrieved data including at least one medicine name,
wherein selecting the subset of items further comprises processing items of commentary which include at least one said at least one medicine name.

18. A method according to claim 1, wherein said plurality of items are initially selected by performing a plurality of searching or filtering operations to find a respective plurality of sets of items of commentary, and forming the plurality of items from the plurality of sets of items of commentary.

19. A method according to claim 17, wherein the searching or filtering operations include searching or filtering by medicine name and additionally by at least one of: commentary type, author type, type of medicine, ailment, condition, symptom, potential side effect, possible medical outcome, treatment type, an aspect of personal experience, measure of the tone of the commentary, detected stance of the patient, and feeling or emotion.

20. A method according to claim 1, wherein processing the content analysis data further comprises:
processing a plurality of feature indicators selected from at least one of: at least one aspect of a personal experience; at least one measure of the commentary, at least one detected stance of the patient, a count of the number of times a medicine is mentioned, a count of the number of times a relevant symptom is mentioned, a sentiment estimate, and a count of the number of times a relevant feeling or experience is mentioned; and
combining the indicators to generate the estimate indicative of the overall effectiveness or safety of the medicine.

21. A method according to claim 20, wherein calculating the estimate indicative of the overall effectiveness or safety of the medicine comprises selecting a set of the feature indicators, applying a respective weighting to each selected feature indicator, and combining the weighted plurality of feature indicators into a single estimate indicative of the overall effectiveness or safety of the medicine.

22. A method according to claim 21, wherein the selected set of feature indicators includes at least one primary indicator of effectiveness or safety of the medicine and at least one relevance indicator, representing a measure of the amount of opinion expressed.

23. A method according to claim 21, further comprising restricting at least one of the selected feature indicators to a sub-range within the output range of the single estimate.

24. A method according to claim 20, wherein processing the content analysis data is carried out with respect to a predetermined time period, wherein the subset of items is selected in respect of commentary falling within the predetermined time period.

25. A method according to claim 1, further comprising:
selecting one of the plurality of items of commentary;
providing annotation data associated with the selected item of commentary, the annotation data identifying at least one of: commentary type, author type, name of medicine, type of medicine, ailment, condition, symptom, potential side effect, possible medical outcome, treatment type, an aspect of a personal experience, measure of the tone of the commentary, detected stance of the patient, and feeling or emotion; and
training or retraining said at least one classifier using the combination of the selected item of commentary and the associated annotation data.

26. A method according to claim 25, further comprising:
outputting the selected item of commentary to an annotation user;
outputting the annotation data to the annotation user;
receiving user input from the annotation user including a direction to create, modify or delete at least a portion of the annotation data; and
creating, modifying or deleting at least a portion of the annotation data in accordance with the received direction.

27. A method according to claim 25, further comprising
processing the commentary data for the selected item using said at least one classifier; and
wherein providing the annotation data includes providing, at least in part, the output of said at least one classifier.

28. A method according to claim 25, further comprising repeating at least one of the steps of: processing the commentary data using said at least one classifier, selecting a subset of items, processing the subset of items, and processing the content analysis data after training or retraining said at least one classifier with the new or modified annotation data.

29. A method of training a selected classifier for use with a method as claimed in claim 1, wherein the selected classifier is configured to identify, for an item of commentary, at least one of a commentary type and a list of medicines associated with the commentary, and wherein the method comprises:
processing the commentary data using at least one classifier, including said selected classifier, to identify for each item a commentary type and a list of medicines associated with by the commentary;
selecting one of the plurality of items of commentary;
providing annotation data associated with the selected item of commentary, the annotation data identifying at least one of: commentary type, author type, name of medicine, type of medicine, ailment, condition, symptom, potential side effect, possible medical outcome, treatment type, an aspect of a personal experience, measure of the tone of the commentary, detected stance of the patient, and feeling or emotion; and
training or retraining the selected classifier using the combination of the selected item of commentary and the associated annotation data.

30. A method according to claim 29, further comprising:
outputting the selected item of commentary to an annotation user;
outputting the annotation data to the annotation user;
receiving user input from the annotation user including a direction to create, modify or delete at least a portion of the annotation data; and
creating, modifying or deleting at least a portion of the annotation data in accordance with the received direction.

31. A method according to claim 29, further comprising
processing the commentary data for the selected item using the selected classifier; and
wherein providing the annotation data includes providing, at least in part, the output of the selected classifier.

32. A computer system for estimating the effectiveness or safety of a medicine, the computer system comprising:
at least one processor and at least one associated memory store;
wherein said at least one memory store includes computer program code which, when executed by said at least one processor, causes the computer system to perform the method of:
receiving commentary data encoding a plurality of items of commentary substantially related to medical subject-matter;
processing the commentary data using at least one classifier to identify for each item a commentary type and a list of medicines associated with the commentary;
selecting a subset of items, from the plurality of items of commentary, identified as referencing the medicine and whose commentary type has been identified as commentary from a patient who has used the medicine;
processing the subset of items to generate content analysis data including, for each item, at least one quantified estimate of at least one aspect of a patient experience described in the commentary; and
processing the content analysis data to calculate an estimate indicative of the overall effectiveness or safety of the medicine.

33. A computer system for training or retraining a classifier for use with a computer system as claimed in claim 32, wherein the selected classifier is configured to identify, for an item of commentary, at least one of a commentary type and a list of medicines associated with the commentary, and wherein the system comprises:
at least one processor and at least one associated memory store;
wherein said at least one memory store includes computer program code which, when executed by said at least one processor, causes the computer system to perform the method of:
selecting one of a plurality of items of commentary;
causing the commentary data for the selected item of commentary to be processed using the selected classifier;
outputting the selected item of commentary to an annotation user;
receiving annotation data associated with the selected item of commentary, the annotation data identifying at least one of: commentary type, author type, name of medicine, type of medicine, ailment, condition, symptom, potential side effect, possible medical outcome, treatment type, an aspect of a personal experience, measure of the tone of the commentary, detected stance of the patient, and feeling or emotion, and the annotation data including, at least in part, the output of the selected classifier;
outputting the annotation data to the annotation user;
receiving user input from the annotation user including a direction to create, modify or delete at least a portion of the annotation data;
creating, modifying or deleting at least a portion of the annotation data in accordance with the received direction; and
causing the selected classifier to be trained or retrained using the combination of the selected item of commentary and the associated annotation data.

34. A computer system for estimating the effectiveness or safety of medicines, the computer system including:
a commentary downloader module for downloading items of commentary from at least one remote source;
a commentary type classifier module for identifying the type of commentary;
a named entity recognition, NER, classifier module for identifying entities associated with each item of commentary;
a medicine database encoding medicine data that encodes a plurality of medicine names associated with at least one jurisdiction;
a commentary importer module which accesses and applies the commentary type classifier module, the NER classifier module and the medicine data in the medicine database, to select from the downloaded items of commentary a plurality of items of commentary that include at least one medicine entity, that include at least one appropriate medicine name, and that are identified as being a commentary type that is authored by a patient;
a feature calculator module configured to calculate for each item of commentary a plurality of feature indicators selected from at least one of: at least one aspect of a personal experience, at least one measure of the tone of the item of commentary, at least one detected stance of the patient, a count of the number of times a medicine is mentioned, a count of the number of times a relevant symptom is mentioned, a sentiment estimate, and a count of the number of times a relevant feeling or experience is mentioned;
a summary score calculator module for calculating a summary score representative of the effectiveness or safety of a medicine in dependence on the feature indicators calculated by the feature calculator module for relevant items of the plurality of items of commentary.

35. A computer system according to claim 34, wherein the sentiment estimate encodes a measure of at least one of: positivity, negativity and neutrality, expressed by the patient in relation to at least one of: the commentary considered as a whole, each mention of the medicine individually, and every mention of the medicine considered as a whole.

36. A computer system according to claim 34, further comprising an annotation entry system configured to:
receive an item of commentary;
receive associated annotation data encoding the output of the commentary type classifier module and the NER classifier module in respect of the item of commentary;
output the item of commentary and the associated annotation data;
receive adjustments or additions to the annotation data;
carry out the adjustments or additions to the annotation data;
transmit the adjusted annotation data and cause at least one of the commentary type classifier module and the NER classifier module to be trained or retrained using the adjusted annotation data.

37. A method of estimating the effectiveness or safety of medicines, comprising:
downloading items of commentary from at least one remote source;
applying a commentary type classifier and a named entity recognition, NER, classifier to select from the downloaded commentary a plurality of items of commentary that include at least one medicine entity and that are identified as being a commentary type that is authored by a patient;
for each of the plurality of items of commentary, calculating a plurality of feature indicators selected from at least one of: at least one aspect of a personal experience, at least one measure of the tone of the item of commentary, at least one detected stance of the patient, a count of the number of times a medicine is mentioned, a count of the number of times a relevant symptom is mentioned, a sentiment estimate, and a count of the number of times a relevant feeling or experience is mentioned; and
calculating a summary score representative of the effectiveness or safety of a medicine in dependence on the feature indicators calculated for relevant items of the plurality of items of commentary.

38. A method according to claim 37, further comprising:
receiving an item of commentary;
receiving associated annotation data encoding the output of the commentary type classifier and the NER classifier in respect of the item of commentary;
outputting the item of commentary and the associated annotation data;
receiving adjustments or additions to the annotation data;
carrying out the adjustments or additions to the annotation data;
transmitting the adjusted annotation data and cause at least one of the commentary type classifier and the NER classifier to be trained or retrained using the adjusted annotation data.

39. A non-transitory computer readable medium encoding computer program code which, when executed on at least one processor of a computer, causes the computer to carry out the method of claim 1.
